(19) United States
(12) Patent Application Publication
(10) Pub. No.: US 2023/0332103 A1
(43) Pub. Date: Oct. 19, 2023

(54) Methods and Compositions for Generating Human Forebrain Neural Progenitor Cells and for Maturation Thereof to Parvalbumin+ Interneurons

(71) Applicant: Trailhead Biosystems Inc., Cleveland, OH (US)

(72) Inventors: Nooshin AMINI, Cleveland, OH (US); Xiaoguang FANG, Solon, OH (US)

(21) Appl. No.: 17/896,781

(22) Filed: Aug. 26, 2022

Related U.S. Application Data
(60) Provisional application No. 63/391,206, filed on Jul. 21, 2022.
(60) Provisional application No. 63/273,742, filed on Oct. 29, 2021.

(51) Int. Cl. C12N 5/0797; C12N 5/073

(52) U.S. Cl. CPC C12N 5/0623; C12N 5/0605; C12N 2501/155; C12N 2501/415

(57) ABSTRACT

Methods for generating human forebrain neural progenitor cells and mature forebrain neurons from human pluripotent stem cells are provided using chemically-defined culture media. The methods allow for the initial generation of OTX2+FEZF2+SIX3+ forebrain neural stem cells, with further culturing allowing for the generation of NKX2-1+ ventral forebrain neural stem cells and subsequently the generation of ASCL1+ medial ganglionic eminence neural progenitor cells (MGE-NPCs). The MGE-NPCs are further differentiated to immatured neurons and mature GABAergic interneurons. Culture media, isolated cell populations and kits are also provided.

### RELATED APPLICATIONS

This application claims priority to U.S. Provisional Application No. 63/273,742, filed Oct. 29, 2021, and U.S. Provisional Application No. 63/391,206 filed Jul. 21, 2022. The entire contents of the prior applications are hereby incorporated by reference in their entirety.

### GOVERNMENT LICENSED RIGHTS

This invention was made with government support under Grant Number: W911NF-17-3-0003 awarded by the U.S. ARMY ACC-AGP-RTP. The government has certain rights in the invention.

### BACKGROUND OF THE INVENTION

The forebrain is an essential functional region of the central nervous system (CNS). Dysfunction of forebrain neurons can lead to severe neurological diseases. GABAergic interneurons of the forebrain are the major inhibitory neurons of the CNS. GABAergic interneurons contribute to key aspects of the functional maturation of the cortex. Cortical GABA interneurons originate from the medial ganglionic eminence (MGE), a ventral forebrain territory. Dysfunction of GABAergic interneurons has been reported to result in neurodegenerative or psychiatric diseases (Fishell and Rudy (2011) Annu. Rev. Neurosci. 34:535:567; Le Magueresse and Monyer (2013) Neuron 77:388-405). The ability to obtain neural progenitor cells from human pluripotent stem cells (hPSCs) that are committed to the forebrain lineage, including those than can develop into GABAergic interneurons, thus provides a means for potentially treating such forebrain-related neurological and psychiatric diseases.

Early approaches for obtaining neural progenitor cells from hPSCs used feeder cell layers and culture media containing serum and/or other undefined components, which is unsuitable for clinical use. More recently, chemically defined feeder-free systems have been developed. For example, an approach has been reported in which hPSCs are first differentiated to induce forebrain neuroepithelial cells using an embryoid body protocol, which requires ten days, followed by culture of the resulting cells with an SHH agonist, which leads to NKX2-1-expressing MGE progenitors and maturation of GABinterneuron subtypes over the course of several weeks (Liu et al. (2013) Nat Protoc. 8:1670-1679; Yuan et al. (2015) Sci Rep 5:18550). Additional forebrain differentiation protocols have been reported in the art, including approaches referred to as the adherent differentiation protocol (Yan et al. (2013) Stem Cells Transl. Med. 2:862-870) and the nonadherent differentiation protocol (Crompton et al. (2013) Stem Cell Res. 11:1206-1221).

Wnt inhibition has been incorporated into strategies for differentiation forebrain neural progenitors and the timing of Wnt inhibition has been reported to modulate the differentiation of medial ganglionic eminence progenitors of GABAergic interneurons (Ihnatovych et al. (2018) Stem Cells International, vol. 2018, Article ID 3983090).

More recently, feeder-free protocols using additional chemical components have been developed. One approach involves an eight day neural induction phase leading to neuroectodermal progenitors (rosettes) followed by a further eight day regionalization phase leading to forebrain progenitors by day 16 (Comella-Bolla et al. (2020) Mol. Neurobiol. 57:2766-2798).

Additional protocols for differentiation of forebrain neural progenitors include use of SMAD 2/3 inhibition, such as by inclusion of a TGFβ antagonist in the culture media (see e.g., Nicholas et al. (2013) Cell Stem Cell 12:573-586; U.S. Patent Publication No. 2016/0272940; U.S. Patent Publication No. 2019/0062700; U.S. Patent Publication No. 2021/0040443).

Additional protocols for differentiation of forebrain neural progenitors are also described in U.S. Patent Publication No. 2015/0361393 and U.S. Patent Publication No. 2017/0292112.

Accordingly, while some progress has been, there remains a need for efficient and robust methods and compositions for generating forebrain-committed neural progenitor cells and mature GABAergic interneurons from human pluripotent stem cells.

### SUMMARY OF THE INVENTION

This disclosure provides methods of generating human forebrain neural progenitor cells, including forebrain neural stem cells (FB-NSCs), ventral forebrain neural stem cells (VFB-NSCs) and medial ganglionic eminence neural progenitor cells (MGE-NPCs), in a three stage protocol requiring nine days, wherein the MGE-NPCs can be further differentiated into mature GABAergic interneurons in a two stage protocol requiring 17 additional days. The methods use chemically-defined culture media that allows for generation of FB-NSCs within three days of culture, VFB-NSCs within six days of culture, MGE-NPCs within nine days of culture, immatured neurons within 12 days of culture and mature parvalbumin positive interneurons within 26 days of culture. The defined culture media used to obtained the different types of neural progenitor cells and matured neurons comprise small molecule agents that either agonize or antagonize particular signaling pathway activity in the pluripotent stem cells such that differentiation along the forebrain neural lineage is promoted, leading to cellular maturation and expression of forebrain neural progenitor-associated biomarkers. The methods of the disclosure use culture media for differentiation that comprise different components than those of earlier protocols, avoiding the need for certain reagents (e.g., the methods of the disclosure do not require dual SMAD inhibition). The methods of the disclosure also have the advantage that the use of small molecule agents in the culture media allows for precise control of the culture components, the need for a neural induction phase is avoided and the time needed for differentiation to MGE-NPCs, followed by further maturation to mature GABAergic interneurons, is significantly shortened compared to prior art protocols.

Accordingly, in one aspect, the disclosure pertains to a method of generating human OTX2+FEZF2+SIX3+ forebrain neural stem cells (FB-NSCs) comprising: culturing human pluripotent stem cells in a culture media comprising a BMP pathway antagonist, a MEK pathway antagonist, a WNT pathway antagonist, an AKT pathway antagonist, an SHH pathway agonist and a PKC pathway antagonist on days 0-3 to obtain human OTX2+FEZF2+SIX3+FB-NSCs.

The method can further comprise further culturing the FB-NSCs on days 3-6 in a culture comprising a BMP pathway antagonist, a MEK pathway antagonist, a WNT pathway antagonist and an SHH pathway agonist to obtain human NKX2-1+ ventral forebrain neural stem cells (VFB-NSCs).

The method can further comprise further culturing the VFB-NSCs on days 6-9 in a culture media comprising a TAK1 pathway antagonist, an SHH pathway agonist, a TGF-β pathway antagonist, a TRK pathway antagonist, a Notch pathway antagonist and an IGF1 pathway agonist to obtain human ASCL1+ medial ganglionic eminence neural progenitor cells (MGE-NPCs).

The method can further comprise further culturing the MGE-NPCs on days 9-12 in a culture media comprising a CREB or PKA pathway agonist, valproic acid or analog, Substance P or analog, a GDNF pathway agonist and an mTOR pathway agonist to obtain human immatured neurons.

The method can further comprise further culturing the human immatured neurons on days 12-26 in a culture media comprising a BDNF pathway agonist, an IGF-1 pathway agonist, ascorbic acid or analog, sodium pyruvate or analog, LPA or analog, an N2 supplement and an NEAA supplement to obtain mature parvalbumin+interneurons.

In an embodiment, the human pluripotent stem cells are induced pluripotent stem cells (iPSCs). In an embodiment, the human pluripotent stem cells are embryonic stem cells. In an embodiment, the human pluripotent stem cells are attached to vitronectin-coated plates during culturing.

In an embodiment, the BMP pathway antagonist is selected from the group consisting of LDN193189, DMH1, DMH2, Dorsopmorphin, K02288, LDN214117, LDN212854, folistatin, ML347, Noggin, and combinations thereof. In an embodiment, the BMP pathway antagonist is present in the culture media at a concentration within a range of 100-500 nM. In an embodiment, the BMP pathway antagonist is LDN193189, which is present in the culture media at a concentration of 250-275 nM.

In an embodiment, the MEK pathway antagonist is selected from the group consisting of PD0325901, Binimetinib (MEK162), Cobimetinib (XL518), Selumetinib, Trametinib (GSK1120212), CI-1040 (PD-184352), Refametinib, ARRY-142886 (AZD-6244), PD98059, U0126, BI-847325, RO 5126766, and combinations thereof. In an embodiment, the MEK pathway antagonist is present in the culture media at a concentration within a range of 50-150 nM. In an embodiment, the MEK pathway antagonist is PD0325901, which is present in the culture media at a concentration of 100-110 nM.

In an embodiment, the WNT pathway antagonist is selected from the group consisting of XAV939, ICG001, Capmatinib, endo-IWR-1, IWP-2, IWP-4, MSAB, CCT251545, KY02111, NCB-0846, FH535, LF3, WIKI4, Triptonide, KYA1797K, JW55, JW 67, JW74, Cardionogen 1, NLS-StAx-h, TAK715, PNU 74654, iCRT3, WIF-1, DKK1, and combinations thereof. In an embodiment, the WNT pathway antagonist is present in the culture media at a concentration within a range 50-150 nM. In an embodiment, the WNT pathway antagonist is XAV939, which is present in the culture media at a concentration of 100-110 nM.

In an embodiment, the SHH pathway agonist is selected from the group consisting of Purmorphamine, GSA 10, SAG, and combinations thereof. In an embodiment, the SHH pathway agonist is present in the culture media at a concentration within a range of 250-750 nM. In an embodiment, the SHH pathway agonist is Purmorphamine, which is present in the culture media at a concentration of 500-550 nM.

In an embodiment, the AKT pathway antagonist is selected from the group consisting of MK2206, GSK690693, Perifosine (KRX-0401), Ipatasertib (GDC-0068), Capivasertib (AZD5363), PF-04691502, AT 7867, Triciribine (NSC154020), ARQ751, Miransertib (ab235550), Borussertib, Cerisertib, and combinations thereof. In an embodiment, the AKT pathway antagonist is present in the culture media at a concentration within a range of 50-200 nM. In an embodiment, the AKT pathway antagonist is MK2206, which is present in the culture media at a concentration of 138 nM.

In an embodiment, the PKC pathway antagonist is selected from the group consisting of Go 6983, Sotrastaurin, Enzastaurin, Staurosporine, LY31615, Go 6976, GF 109203X, Ro 31-8220 Mesylate, and combinations thereof. In an embodiment, the PKC pathway antagonist is present in the culture media at a concentration within a range of 50-200 nM. In an embodiment, the PKC pathway antagonist is Go 6983, which is present in the culture media at a concentration of 110 nM.

In an embodiment, the TAK1 pathway antagonist is selected from the group consisting of Takinib, Dehydoabietic acid, NG25, Sarsasapogenin, and combinations thereof. In an embodiment, the TAK1 pathway antagonist is present in the culture media at a concentration within a range of 1-5 uM. In an embodiment, the TAK1 pathway antagonist is Takinib, which is present in the culture media at a concentration of 2 uM.

In an embodiment, the TGFβ pathway antagonist is selected from the group consisting of A 83-01, SB-431542, GW788388, SB525334, TP0427736, RepSox, SD-208, and combinations thereof. In an embodiment, the TGFβ pathway antagonist is present in the culture media at a concentration within a range of 250-750 nM. In an embodiment, the TGFβ pathway antagonist is A 83-01, which is present in the culture media at a concentration of 500 nM.

In an embodiment, the TRK pathway antagonist is selected from the group consisting of GNF-5837, BMS-754807, UNC2020, Taletrectinib, Altiratinib, Selitrectinib, PF 06273340, and combinations thereof. In an embodiment, the TRK pathway antagonist is present in the culture media at a concentration within a range of 25-75 nM. In an embodiment, the TRK pathway antagonist is GNF-5837, which is present in the culture media at a concentration of 50 nM.

In an embodiment, the Notch pathway antagonist is selected from the group consisting of GSI-XX, RO4929097, Semagacestat, Dibenzazepine, LY411575, Crenigacestat, IMR-1, IMR-1A, FLI-06, DAPT, Valproic acid, YO-01027, CB-103, Tangeretin, BMS-906024, Avagacestat, Bruceine D, and combinations thereof. In an embodiment, the Notch pathway antagonist is present in the culture media at a concentration within a range of 50-150 nM. In an embodiment, the Notch pathway antagonist is GSI-XX, which is present in the culture media at a concentration of 100 nM.

In an embodiment, the IGF1 pathway agonist is selected from the group consisting of IGF1, IGF1-Ado, X10, mecasermin, and combinations thereof. In an embodiment, the IGF1 pathway agonist is present in the culture media at a concentration within a range of 5-15 ng/ml.

In an embodiment, the IGF1 pathway agonist is IGF1, which is present in the culture media at a concentration of 10 ng/ml.

In an embodiment, the CREB or PKA pathway agonist is selected from the group consisting of cAMP, Dibutyryl-cAMP, 8-Br-cAMP, cAMPS-Sp, CW 008, Forskolin, 8-CPT-cAMP, CW 008, Adenosine 3′,5′-cyclic Monophosphate, N6-Benzoyl-, Sodium Salt, Adenosine 3′,5′-cyclic monophosphate sodium salt monohydrate, (S)-Adenosine, cyclic 3prime,5prime-(hydrogenphosphorothioate) triethylammonium, Sp-Adenosine 3prime,5prime-cyclic monophosphorothioate triethylammonium salt, Sp-5,6-DCI-cBiMPS, 8-Bromoadenosine 3′,5′-cyclic Monophosphothioate, Sp-Isomer sodium salt, Adenosine 3prime,5prime-cyclic Monophosphorothioate,8-Bromo-, Sp-Isomer, Sodium Salt, Sp-8-pCPT-cyclic GMPS Sodium, 8-Bromoadenosine 3′,5′-cyclic monophosphate, N6-Monobutyryladenosine 3prime:5prime-cyclic monophosphate sodium salt, 8-PIP-cAMP, Sp-cAMPS, and combinations thereof. In an embodiment, the CREB or PKA pathway agonist is present in the culture media at a concentration within a range of 0.5-2.5 μM. In an embodiment, the CREB or PKA pathway agonist is cAMP, which is present in the culture media at a concentration of 1.0-1.5 μM.

In an embodiment, valproic acid or analog is selected from the group consisting of valproic acid, valproate, sodium valproate and valproate semisodium. In an embodiment, valproic acid or analog is present in the culture media at a concentration within a range of 250-750 nM. In an embodiment, valproic acid is present in the culture media at a concentration within a range of 450-550 nM.

In an embodiment, Substance P is present in the culture media at a concentration within a range of 100-150 nM.

In an embodiment, the GDNF pathway agonist is selected from the group consisting of GDNF, BT13, BT44, and combinations thereof. In an embodiment, the GDNF pathway agonist is present in the culture media at a concentration within a range of 5-50 ng/ml. In an embodiment, the GDNF pathway agonist is GDNF, which is present in the culture media at a concentration of 10 ng/ml.

In an embodiment, the mTOR pathway agonist is selected from the group consisting of MHY1458, NV-5138, Testosterone, 3-benzyl-5-((2-nitrophenoxy) methyl)-dihydrofuran-2(3H)-one (3BDO), 3BDO, L-leucine, NV-5138 hydrochloride, NV-5138, L-leucine-d1, L-leucine-2-13C,15N, Leucine-13C6, L-leucine-d7, L-leucine-d10, L-leucin-d2, l-leucine-d3, L-leucine-18O2, L-leucine-13C, L-leucine-2-13C, L-leucine-13C6-15N, L-leucine-15N, L-leucine-1-13C,15N, and combinations thereof. In an embodiment, the mTOR pathway agonist is present in the culture media at a concentration within a range of 1-3 μM. In an embodiment, the mTOR agonist is MHY1458, which is present in the culture media at a concentration of 2 μM.

In an embodiment, the BDNF pathway agonist is selected from the group consisting of BDNF, rotigotine, 7,8-DHF, ketamine, tricyclic dimeric peptide-6 (TDP6), LM22A-4, and combinations thereof. In an embodiment, the BDNF pathway agonist is present in the culture media at a concentration within a range of 5-50 ng/ml. In an embodiment, the BDNF pathway agonist is BDNF, which is present in the culture media at a concentration of 10 ng/ml.

In an embodiment, ascorbic acid or analog thereof is selected from the group consisting of vitamin C, 2-phospho-L-ascorbic acid, L-ascorbic acid, sodium ascorbyl phosphate, magnesium ascorbyl phosphate, ascorbyl glucoside, tetrahexyldecyl ascorbate (THD), ethylated L-ascorbic acid, and combinations thereof. In an embodiment, ascorbic acid or analog is present in the culture media at a concentration within a range of 100-400 μM. In an embodiment, 2-phospho-L-ascorbic acid is present in the culture media at a concentration of 200 μM.

In an embodiment, sodium pyruvate is present in the culture media at a concentration of 100-300 μM.

In an embodiment, LPA or analog thereof is selected from the group consisting of lysophosphatidic acid (LPA), 2-[[3-(1,3-dioxo-1H-benz[de]isoquinolin-2(3H)-yl)propyl]thio]benzoic acid, 1-Oleoyl lysophosphatidic acid (O-LPA), UCM-05194, and combinations thereof. In an embodiment, LPA or analog is present in the culture media at a concentration within a range of 100-400 nM. In an embodiment, LPA or analog is O-LPA, which is present in the culture media at a concentration within a range of of 200-300 nM.

In an embodiment, the N2 supplement is present in the culture media at a concentration in a range of 0.5%-1.5%.

In an embodiment, the NEAA supplement is present in the culture media at a concentration in a range of 0.5%-1.5%.

In another aspect, the disclosure provides a method of generating human NKX2-1 ventral forebrain neural stem cells (VFB-NSCs) comprising:
(a) culturing human pluripotent stem cells in a culture media comprising a BMP pathway antagonist, a MEK pathway antagonist, a WNT pathway antagonist, an AKT pathway antagonist, an SHH pathway agonist and a PKC pathway antagonist on days 0-3 to obtain human OTX2+FEZF2+SIX3+ forebrain neural stem cells (FB-NSCs); and
(b) further culturing the human OTX2+FEZF2+SIX3+FB-NSCs on days 3-6 in a culture media comprising a BMP pathway antagonist, a MEK pathway antagonist, a WNT pathway antagonist and an SHH pathway agonist and lacking an AKT pathway antagonist and a PKC pathway antagonist to obtain human NKX2-1+ ventral forebrain neural stem cells (VFB-NSCs).

In an embodiment, the BMP pathway antagonist is LDN193189, the MEK pathway antagonist is PD0325901, the WNT pathway antagonist is XAV939, the SHH pathway agonist is Purmorphamine, the AKT pathway antagonist is MK2206 and the PKC pathway antagonist is Go 6983.

In an embodiment, LDN193189 is present in the culture media at a concentration within a range of 100-500 nM, PD0325901 is present in the culture media at a concentration within a range of 50-150 nM, XAV939 is present in the culture media at a concentration within a range of 50-150 nM, Purmorphamine is present in the culture media at a concentration within a range of 250-750 nM, MK2206 is present in the culture media in step (a) at a concentration within a range of 50-150 nM, and Go 6983 is present in the culture media in step (a) at a concentration within a range of 50-150 nM. In an embodiment, LDN193189 is present in the culture media at a concentration of 275 nM in step (a) and 250 nM in step (b), PD0325901 is present in the culture media at a concentration of 110 nM in step (a) and 100 nM in step (b), XAV939 is present in the culture media at a concentration of 110 nM in step (a) and 100 nM in step (b), Purmorphamine is present in the culture media at a concentration of 550 nM in step (a) and 500 nM in step (b), MK2206 is present in the culture media in step (a) at a concentration of 138 nM, and Go 6983 is present in the culture media in step (a) at a concentration of 110 nM.

In yet another aspect, the disclosure pertains to a method of generating human ASCL1+ medial ganglionic eminence neural progenitor cells (MGE-NPCs) comprising:
(a) culturing human pluripotent stem cells in a culture media comprising a BMP pathway antagonist, a MEK pathway antagonist, a WNT pathway antagonist, an AKT pathway antagonist, an SHH pathway agonist and a PKC pathway antagonist on days 0-3 to obtain human OTX2+FEZF2+SIX3+ forebrain neural stem cells (FB-NSCs);
(b) further culturing the human OTX2+FEZF2+SIX3+FB-NSCs on days 3-6 in a culture media comprising a BMP pathway antagonist, a MEK pathway antagonist, a WNT pathway antagonist and an SHH pathway agonist and lacking an AKT pathway antagonist and a PKC pathway antagonist to obtain human NKX2-1+ ventral forebrain neural stem cells (VFB-NSCs); and
(c) further culturing the human NKX2-1+VFB-NSCs on days 6-9 in a culture media comprising a TAK1 pathway antagonist, an SHH pathway agonist, a TGF-β pathway antagonist, a TRK pathway antagonist, a Notch pathway antagonist and an IGF1 pathway agonist to obtain human ASCL1+ medial ganglionic eminence neural progenitor cells (MGE-NPCs).

In an embodiment, the BMP pathway antagonist is LDN193189, the MEK pathway antagonist is PD0325901, the WNT pathway antagonist is XAV939, the SHH pathway agonist is Purmorphamine, the AKT pathway antagonist is MK2206, the PKC pathway antagonist is Go 6983, the TAK1 pathway antagonist is Takinib, the TGF-β pathway antagonist is A 83-01, the TRK pathway antagonist is GNF-5837, the Notch pathway antagonist is GSI-XX and the IGF1 pathway agonist is IGF-1.

In an embodiment, LDN193189 is present in the culture media at a concentration within a range of 100-500 nM, PD0325901 is present in the culture media at a concentration within a range of 50-150 nM, XAV939 is present in the culture media at a concentration within a range of 50-150 nM, Purmorphamine is present in the culture media at a concentration within a range of 250-750 nM, MK2206 is present in the culture media in step (a) at a concentration within a range of 50-150 nM, Go 6983 is present in the culture media in step (a) at a concentration within a range of 50-150 nM, Takinib is present in the culture media in step (c) at a concentration within a range of 1-5 uM, A 83-01 is present in the culture media in step (c) at a concentration within a range of 250-750 nM, GNF-5837 is present in the culture media in step (c) at a concentration within a range of 25-75 nM, GSI-XX is present in the culture media in step (c) at a concentration within a range of 50-150 nM and IGF-1 is present in the culture media in step (c) at a concentration within a range of 5-15 ng/ml. In an embodiment, LDN193189 is present in the culture media at a concentration of 275 nM in step (a) and 250 nM in step (b), PD0325901 is present in the culture media at a concentration of 110 nM in step (a) and 100 nM in step (b), XAV939 is present in the culture media at a concentration of 110 nM in step (a) and 100 nM in step (b), Purmorphamine is present in the culture media at a concentration of 550 nM in step (a) and 500 nM in step (b), MK2206 is present in the culture media in step (a) at a concentration of 138 nM, Go 6983 is present in the culture media in step (a) at a concentration of 110 nM, Takinib is present in the culture media in step (c) at a concentration of 2 uM, A 83-01 is present in the culture media in step (c) at a concentration of 500 nM, GNF-5837 is present in the culture media in step (c) at a concentration of 50 nM, GSI-XX is present in the culture media in step (c) at a concentration of 100 nM and IGF-1 is present in the culture media in step (c) at a concentration within a range of 10 ng/ml.

In yet another aspect, the disclosure pertains to a method of generating human mature parvalbumin+interneurons from human medial ganglionic eminence neural progenitor cells (MGE-NPCs) comprising:
(a) culturing human MGE-NPCs in a culture media comprising a CREB or PKA pathway agonist, valproic acid or analog, Substance P or analog, a GDNF pathway agonist and an mTOR pathway agonist on days 0-3 to obtain human immatured neurons; and
(b) culturing the human immatured neurons in a culture media comprising a BDNF pathway agonist, an IGF-1 pathway agonist, ascorbic acid or analog, sodium pyruvate or analog, LPA or analog, an N2 supplement and an NEAA supplement on days 3-17 to obtain human mature parvalbumin+interneurons.

In an embodiment, the CREB or PKA pathway agonist is cAMP, the valproic acid or analog is valproic acid, the Substance P or analog is Substance P, the GDNF pathway agonist is GDNF, the mTOR pathway agonist is MHY1458, the BDNF pathway agonist is BDNF, the IGF-1 pathway agonist is IGF-1, the ascorbic acid or analog is 2-phospho-L-ascorbic acid, the sodium pyruvate or analog is sodium pyruvate and the LPA or analog is O-LPA.

In an embodiment, cAMP is present at a concentration in a range of 1.0-1.5 μM, valproic acid is present at a concentration in a range of 450-550 nM, Substance P is present at a concentration in a range of 100-150 nM, GDNF is present at a concentration in a range of 5-50 ng/ml, MHY1458 is present at a concentration in a range of 1-3 μM, BDNF is present at a concentration in a range of 5-50 ng/ml, IGF-1 is present at a concentration in a range of 5-50 ng/ml, 2-phospho-L-ascorbic acid is present at a concentration in a range of 100-400 μM, 0-LPA is present at a concentration in a range of 100-400 μM, sodium pyruvate is present at a concentration in a range of 100-300 μM; N2 supplement is present at a concentration in a range of 0.5%-1.5% and NEAA supplement is present at a concentration in a range of 0.5%-1.5%.

In an embodiment, cAMP is present at a concentration of 1.0 μM, valproic acid is present at a concentration of 500 nM, Substance P is present at a concentration of 100 nM, GDNF is present at a concentration of 10 ng/ml, MHY1458 is present at a concentration of 2 μM, BDNF is present at a concentration of 10 ng/ml, IGF-1 is present at a concentration of 10 ng/ml, 2-phospho-L-ascorbic acid is present at a concentration of 200 μM, sodium pyruvate is present at a concentration of 100 μM, 0-LPA is present at a concentration of 200 μM, N2 supplement is present at a concentration of 1.0% and NEAA supplement is present at a concentration of 1.0%.

In another aspect, the disclosure pertains to various culture media for generating human forebrain neural stem or progenitor cells. In an embodiment, the disclosure provides a culture media for obtaining human OTX2+FEZF2+SIX3+ forebrain neural stem cells (FB-NSCs) comprising a BMP pathway antagonist, a MEK pathway antagonist, a WNT pathway antagonist, an AKT pathway antagonist, an SHH pathway agonist and a PKC pathway antagonist. In an embodiment, the disclosure provides a culture media for obtaining human NKX2-1+ ventral forebrain neural stem cells (VFB-NSCs) comprising a BMP pathway antagonist, a MEK pathway antagonist, a WNT pathway antagonist and an SHH pathway agonist. In an embodiment, the disclosure provides a culture media for obtaining human ASCL1+ medial ganglionic eminence neural progenitor cells (MGE-NPCs) comprising a TAK1 pathway antagonist, an SHH pathway agonist, a TGF-β pathway antagonist, a TRK pathway antagonist, a Notch pathway antagonist and an IGF1 pathway agonist. In an embodiment, the disclosure provides a culture media for obtaining human immatured neurons comprising a CREB or PKA pathway agonist, valproic acid or analog, Substance P or analog, a GDNF pathway agonist and an mTOR pathway agonist. In an embodiment, the disclosure provides a culture media for obtaining human mature GABAergic interneurons comprising a BDNF pathway agonist, an IGF-1 pathway agonist, ascorbic acid or analog, sodium pyruvate or analog, LPA or analog, an N2 supplement and an NEAA supplement.

In another aspect, the disclosure pertains to isolated cell cultures of human forebrain neural stem or progenitor cells. In an embodiment, the disclosure provides an isolated cell culture of human OTX2+FEZF2+SIX3+ forebrain neural stem cells (FB-NSCs), the culture comprising human OTX2+FEZF2+SIX3+FB-NPCs cultured in a culture media comprising a BMP pathway antagonist, a MEK pathway antagonist, a WNT pathway antagonist, an AKT pathway antagonist, an SHH pathway agonist and a PKC pathway antagonist. In an embodiment, the disclosure provides an isolated cell culture of human NKX2-1+ ventral forebrain neural stem cells (VFB-NSCs), the culture comprising human NKX2-1+VFB-NPCs cultured in a culture media comprising a BMP pathway antagonist, a MEK pathway antagonist, a WNT pathway antagonist and an SHH pathway agonist. In an embodiment, the disclosure provides an isolated cell culture of human ASCL1+ medial ganglionic eminence neural progenitor cells (MGE-NPCs), the culture comprising human ASCL1+MGE-NPCs cultured in a culture media comprising a TAK1 pathway antagonist, an SHH pathway agonist, a TGF-β pathway antagonist, a TRK pathway antagonist, a Notch pathway antagonist and an IGF1 pathway agonist. In an embodiment, the disclosure provides an isolated cell culture of human immatured neurons, the culture comprising human immatured neurons cultured in a culture media comprising a CREB or PKA pathway agonist, valproic acid or analog, Substance P or analog, a GDNF pathway agonist and an mTOR pathway agonist. In an embodiment, the disclosure provides an isolated cell culture of human mature GAB Aergic interneurons, the culture comprising human mature GABAergic interneurons cultured in a culture media comprising a BDNF pathway agonist, an IGF-1 pathway agonist, ascorbic acid or analog, sodium pyruvate or analog, LPA or analog, an N2 supplement and an NEAA supplement.

In yet another aspect, the disclosure pertains to human forebrain neural stem or progenitor cells generated by the methods of the disclosure. In an embodiment, the disclosure provides human OTX2+FEZF2+SIX3+ forebrain neural stem cells (FB-NSCs) generated by a method of the disclosure. In an embodiment, the disclosure provides human NKX2-1+ ventral forebrain neural stem cells (VFB-NSCs) generated by a method of the disclosure. In an embodiment, the disclosure provides human ASCL1+ medial ganglionic eminence neural progenitor cells (MGE-NPCs) generated by a method of the disclosure. In an embodiment, the disclosure provides human immatured neurons generated by a method of the disclosure. In an embodiment, the disclosure provides human parvalbumin+mature interneurons generated by a method of the disclosure.

The methods and compositions of the disclosure are useful in generation of human forebrain neural stem or progenitor cells for research or therapeutic purposes, such as in the treatment of neurological disorders (e.g., transplantation for the treatment of epilepsy).

Other features and advantages of the invention will be apparent from the following detailed description and claims.

### BRIEF DESCRIPTION OF THE DRAWINGS

FIG. 1 shows results from an HD-DoE model of a 12-factor experiment optimized for maximum expression of FEZF2. The upper section of the model shows the prediction of expression level of pre-selected 53 genes when optimized for FEZF2. The lower section of the model shows the effectors that were tested in this model and their contribution to maximum expression of FEZF2. The value column refers to required concentration of each effector to mimic the model.

FIG. 2 shows the results from an HD-DoE model of a 12-factor experiment optimized for maximum expression of SIX3. Upper and lower sections are as described for FIG. 1. This condition highlights the effectors LDN193189 and TTNPB with factor contributions of 24.9 and 23.7 as an important input for high expression of SIX3.

FIG. 3 shows the results from an HD-DoE model of a 12-factor experiment optimized for maximum expression of OTX2. Upper and lower sections are as described for FIG. 1. This condition highlights the effector TTNPB with factor contribution of 28.8 as an important input minimum expression of OTX2.

FIG. 4 shows the dynamic profile of expression levels of OTX2, FEZF2 and SIX3 genes relative to the concentration of 12 effectors tested. The positive impact of LDN193189 and XAV939 on expression of OTX2 and their factor contribution is shown by slope of the plots for each effector.

FIG. 5 shows the results from an HD-DoE model of a 12-factor experiment optimized for maximum expression of FEZF2. Upper and lower sections are as described for FIG. 1. This condition highlights the effector MK2206 with factor contribution of 12.2 as an important input for maximum expression of FEZF2.

FIG. 6 shows the dynamic profile of expression levels of FEZF2 relative to the concentration of 12 effectors tested. The positive impact of LDN193189, XAV939, PD0325901, MK2206, Purmorphamine and GO6983 on expression of FEZF2 and their factor contribution is shown by slope of the plots for each effector.

FIG. 7 shows the results from an HD-DoE model of a 12-factor experiment applied on stage 1 neural stem cells to generate a recipe for stage 2 of differentiation. This model is optimized for maximum expression of NKX2-1. Upper and lower sections are as described for FIG. 1. This setting highlights the negative role of SANT-1 in expression of NKX2-1 with factor contribution of 26.3.

FIG. 8 shows the dynamic profile of expression levels of NKX2-1, PAX6 and NKX2-2 genes relative to concentration of 12 effectors. Positive impact of PD0325901 and XAV939 on NKX2-1 and negative impact t of SANT-1 and TTNPB and their factor contribution is shown by slope of the plots for each effector.

FIG. 9 shows the results from an HD-DoE model of a 12-factor experiment applied on stage 2 neural stem cells to generate a recipe for stage 3 of differentiation. This model is optimized for maximum expression of ASCL1. Upper and lower sections are as described for FIG. 1. This setting highlights the positive roles of A8301 and GSI-XX with factor contributions of 18 and 14.

FIG. 10 shows the dynamic profile of expression levels of ASCL1, DLX1 and LHX6 genes relative to concentration of 12 effectors. Positive impact of A8301, GSI-XX and Purmorphamine on expression level of ASCL1 and LHX6 and their factor contribution is shown by slope of the plots for each effector.

FIGS. 11A-11B show the dynamic profile of expression levels of OTX2, FEZF2 and SIX3 genes relative to concentration of 6 validated effectors in recipe of stage 1 of differentiation. FIG. 11A shows expression levels of genes of interest in the presence of all finalized effectors. FIG. 11B shows expression levels of genes of interest in absence of one finalized effector at a time while others are present.

FIG. 12 shows the dynamic profile of expression levels of FEZF2 relative to concentration of 6 validated effectors in recipe of stage 1 of differentiation. The expression level of FEZF2 is shown in the absence of one finalized effector at a time while the others are present.

FIGS. 13A-13B shows the dynamic profile of expression levels of NKX2-1, PAX6 and NKX2-2 genes relative to concentration of 4 validated effectors in recipe of stage 2 of differentiation. FIG. 13A shows expression levels of genes of interest in the presence of all finalized effectors. FIG. 13B shows expression levels of genes of interest in absence of one finalized effector at a time while others are present.

FIG. 14 shows an interaction plot of two effectors in a 12-factor HD-DoE model used for optimization of expression level of NKX2-1. The blue plot shows the level of NKX2-1 expression as the concentration of XAV939 increases while the concentration of Purmorphamine is kept at its highest (500 nM). The green plot shows the level of NKX2-1 expression as the concentration of XAV93 increases while the concentration of Purmorphamine is kept at 0.

FIGS. 15A-15B shows the dynamic profile of expression levels of ASCL1, DLX1 and LHX6 genes relative to concentration of 6 validated effectors in recipe of stage 3 of differentiation. FIG. 15A shows expression levels of genes of interest in presence of all six factors. FIG. 15B shows expression levels of genes of interest in absence of one finalized factor at a time while others are present.

FIGS. 16A-16C shows photographs of fluorescence images of ventral forebrain-derived neural progenitor cells at the end of stage 1, 2 and 3 treatments. Cells are stained with forebrain biomarkers including SIX3, OTX2, dorsal forebrain marker PAX6, ventral forebrain biomarkers NKX2-1, DLX5 and MASH1, MGE specific biomarkers LHX6 and SOX6, pan neuronal biomarker beta III-tubulin, GABAergic interneuron biomarker GABA, proliferation biomarker KI67 and glial biomarkers OLIG2 and GFAP. FIG. 16A shows photographs of fluorescence images of cells at day 3 (end of stage 1). FIG. 16B shows photographs of fluorescence images of cells at day 6 (end of stage 2). FIG. 16C shows photographs of fluorescence images of cells at day 9 (end of stage 3).

FIG. 17 is a schematic diagram of a representative culture method of the disclosure pertaining to the three stage protocol for generating MGE-NPCs from pluripotent stem cells.

FIG. 18 is a schematic diagram of a representative culture method of the disclosure pertaining to the two stage protocol for generating mature GAB Aergic interneurons from MGE-NPCs.

FIG. 19 shows the results from a model of a 12-factor experiment optimized for maximum expression of MEF2C. The upper section of the model shows the prediction of expression level of pre-selected 53 genes when optimized for MEF2C. The lower section of the model shows the effectors that were tested in this model and their contribution to maximum expression of MEF2C. The value column refers to required concentration of each effector to mimic the model.

FIG. 20 shows the dynamic profile of expression levels of MEF2C gene relative to the concentration of 12 effectors. The positive impact of cAMP, Valproic Acid, Substance P, and GDNF on MEF2C and their factor contribution is shown by slope of the plots for each effector.

FIG. 21 shows the results from a model of an 8-factor experiment optimized for maximum expression of PVALB. Upper and lower sections are as described in FIG. 19. This condition highlights the effector, BDNF, IGF-1, and 2-phospho-L-ascorbic acid with factor contribution of 14.6, 12.8, 15.04 as important input for maximum expression of PVALB.

FIG. 22 shows the dynamic profile of expression levels of PVALB relative to the concentration of 8 effectors. The positive impact of Indolactam-V, Oleic acid, 2-phospho-L-Ascorbic acid, BDNF, IGF-1 and their factor contribution is shown by slope of the plots for each effector.

FIG. 23 shows the results from a model of an 8-factor experiment applied on stage 4 neural stem cells to generate a recipe for stage 5 of differentiation. This model is optimized for maximum expression of PVALB. Upper and lower sections are as described in FIG. 19. This setting highlights the positive role of N2 in expression of PVALB with factor contribution of 15.9.

FIG. 24 shows the dynamic profile of expression levels of PVALB genes relative to the concentration of 8 effectors. The positive impact of N2 on PVALB and negative impact of GSI-XX, THI0019 and Heparin and their factor contributions are shown by slope of the plots for each effector.

FIG. 25 shows the results from a model of an 8-factor experiment applied on stage 4 neural stem cells to generate a recipe for stage 5 of differentiation. This model is optimized for maximum expression of PVALB. Upper and lower sections are as described in FIG. 19. This setting highlights the positive roles of Forskolin and Sodium pyruvate with factor contributions of 3.14 and 17.1.

FIG. 26 shows the dynamic profile of expression levels of PVALB gene relative to the concentration of 8 effectors. The positive impact of sodium pyruvate and Forskolin on expression level of PVALB and their factor contributions are shown by slope of the plots for each effector.

FIGS. 27A-27B shows the dynamic profile of expression levels of MEF2C, PVALB and Somatostatin genes relative to the concentration of 5 effectors in recipe of stage 4 of differentiation. Results show expression levels of genes of interest in the presence of all seven factors.

FIGS. 28A-28B shows the dynamic profile of expression levels of PVALB and Somatostatin genes relative to the concentration of 3 effectors in recipe of stage 5 of differentiation. Results show expression levels of genes of interest in the presence of indicated factors.

FIG. 29 shows the dynamic profile of expression levels of PVALB and Somatostatin genes relative to the concentration of N2 effector in recipe of stage 5 of differentiation. The results show expression levels of genes of interest in the presence of N2 factor.

FIG. 30 shows the dynamic profile of expression levels of PVALB and Somatostatin genes relative to concentration of sodium pyruvate in recipe of stage 5 of differentiation. Results show expression levels of genes of interest in the presence of sodium pyruvate factor.

FIGS. 31A-31B shows the dynamic profile of expression levels of LHX6 and GAD1 genes relative to the concentration of effectors in recipe of stage 5 of differentiation. Results show expression levels of genes of interest in the presence of all factors.

FIG. 32 shows photographs of fluorescence images of differentiated interneurons cells at the end of stage 4 treatments. Cells were stained with forebrain biomarkers including NeuN, dorsal forebrain marker PAX6, GAD65, Nestin, MASH1, pan neuronal biomarker beta III-tubulin, SOX2, and SOX6. Results show fluorescence images of cells at day 3 (end of stage 4).

FIG. 33 shows photographs of fluorescence images of differentiated interneurons cells at the end of 5 treatments. Cells were stained with forebrain biomarkers including GAD65, MASH1, MAP2, Synapsin, GABAergic interneuron biomarker GABA, LHX6, Neurofilament, Parvalbumin and GFAP. Results show fluorescence images of cells at day 17 (end of stage 5).

### DETAILED DESCRIPTION OF THE INVENTION

Described herein are methodologies and compositions that allow for the generation of forebrain neural progenitors from human pluripotent stem cells, as well as matured GABAergic interneurons, under chemically-defined culture conditions using a small molecule based approach. The methods of the disclosure generate medial ganglionic eminence neural progenitors (the precursors to GAB Aergic interneurons) in a three stage protocol in which OTX2+FEZF2+SIX3+ forebrain neural stem cells (FB-NSCs) are generated in three days, followed by generation of NKX2-1+ ventral forebrain neural stem cells (VFB-NSCs) by day six of culture, followed by generation of ASCL1+ medial ganglionic eminence neural progenitor cells (MGE-NPCs) by day nine of culture. Thus, the disclosure allows for obtention of MGE-NPCs in a significantly shorter time than prior art protocols using chemically-defined culture conditions. The MGE-NPCs can be further differentiated according to a two stage protocol in which the MGE-NPCs are first differentiated into immatured neurons in three days (day 12 of culture following the nine day protocol to generate MGE-NPCs), followed by differentiation into mature parvalbumin+interneurons in 14 days (day 26 of culture).

As described in Example 1, a High-Dimensional Design of Experiments (HD-DoE) approach was used to simultaneously test multiple process inputs (e.g., small molecule agonists or antagonists) on output responses, such as gene expression. These experiments allowed for the identification of chemically-defined culture media, comprising agonists and/or antagonists of particular signaling pathways, that is sufficient to generate forebrain neural stem and progenitor cells, including FB-NSCs, VFB-NSCs, and MGE-NPCs, under defined conditions in a short amount of time. The optimized culture media was further validated by a factor criticality analysis, which examined the effects of eliminating individual agonist or antagonist agents, as described in Example 2. Immunohistochemistry further confirmed the phenotype of the cells generated by the differentiation protocol, as described in Example 3.

FIG. 17 schematically illustrates an embodiment of the method of the disclosure for generating FB-NSCs, VFB-NSCs and MGE-NPCs using a three stage protocol.

FIG. 18 schematically illustrates an embodiment of the method of the disclosure for generating immatured neurons and mature parvalbumin+interneurons from MGE-NPCs using a two stage protocol.

Various aspects of the invention are described in further detail in the following subsections.

### I. CELLS

The starting cells used in the cultures of the disclosure are human pluripotent stem cells. As used herein, the term “human pluripotent stem cell” (abbreviated as hPSC) refers to a human stem cell that has the capacity to differentiate into a variety of different cell types. The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, for example, using a nude mouse and teratomas formation assay. Pluripotency can also evidenced by the expression of embryonic stem (ES) cell markers, although the preferred test for pluripotency is the demonstration of the capacity to differentiate into cells of each of the three germ layers.

Human pluripotent stem cells include, for example, induced pluripotent stem cells (iPSC) and human embryonic stem cells, such as ES cell lines. Non-limiting examples of induced pluripotent stem cells (iPSC) include 19-11-1, 19-9-7 or 6-9-9 cells (e.g, as described in Yu, J. et al. (2009) Science 324:797-801). Non-limiting examples of human embryonic stem cell lines include ES03 cells (WiCell Research Institute) and H9 cells (Thomson, J. A. et al. (1998) Science 282:1145-1147). Human pluripotent stem cells (PSCs) express cellular markers that can be used to identify cells as being PSCs. Non-limiting examples of pluripotent stem cell markers include TRA-1-60, TRA-1-81, TRA-2-54, SSEA1, SSEA3, SSEA4, CD9, CD24, OCT3, OCT4, NANOG and/or SOX2. Since the methods of generating forebrain neural progenitor of the disclosure are used to differentiate (maturate) the starting pluripotent stem cell population, in various embodiments the forebrain neural progenitor cell populations generated by the methods of the disclosure lack expression of one or more stem cell markers, such as one or more stem cell markers selected from the group consisting of TRA-1-60, TRA-1-81, TRA-2-54, SSEA1, SSEA3, SSEA4, CD9, CD24, OCT3, OCT4, NANOG and/or SOX2.D

The pluripotent stem cells are subjected to culture conditions, as described herein, that induce cellular differentiation. As used herein, the term “differentiation” refers to the development of a cell from a more primitive stage towards a more mature (i.e. less primitive) cell, typically exhibiting phenotypic features of commitment to a particular cellular lineage.

As used herein, a “neural stem cell” refers to a cell that is more differentiated than a pluripotent stem cell in that it is committed to the neural lineage but still has the capacity to differentiate into different types of cells along the neural lineage.

As used herein a “neural progenitor cell” refers to a cell that is more differentiated than a neural stem cell and that can be further differentiated into a particular type of neural cell.

In embodiments, cells can be identified and characterized based on expression of one or more biomarkers, such as particular biomarkers of neural progenitors or forebrain region-committed neural cells. A “positive” biomarker is one that is expressed on a cell of interest, whereas a “negative” biomarker is one that is not expressed on a cell of interest.

Non-limiting examples of biomarkers whose expression can be assessed in the characterization of cells of interest include genes involved in development and patterning of the anterior neuroectoderm and forebrain including OTX2 (Hoch et al. (2015) Cell Reports 12:482-494), SIX3 (Lagutin et al. (2003) Genes & Development 17:368-379) and FEZF2 (Zhang et al. (2014) Development 141:4794-47805), genes expressed in ventral forebrain including NKX2-1 and absence of PAX6 (i.e., PAX6 as a negative biomarker), which is a dorsal forebrain marker (Stoykova et al. (2000) J. Neurosci. 20:8042-8050), and genes expressed in neuronal progenitor cells in medial ganglionic eminence (MGE) region including ASCL1, LHX6 and DLX1 (Silberberg et al. (2016) Neuron 92:59-74). In embodiments, a FB-NSC is OTX2+FEZF2+SIX3+. In embodiments, a VFB-NSC is NKX2-1+. In embodiments, a VFB-NSC is NKX2-1+ and PAX6 negative (PAX6−). In embodiments, an MBE-NPC is ASCL1+. In embodiments, an MBE-NPC is positive for at least two markers selected from ASCL1, LHX6 and DLX1. In embodiments, an MBE-NPC is ASCL1+LHX6+DLX1+.

Other biomarkers included beta III-tubulin (a pan neuronal marker), KI67 (a proliferation marker), DLX5 (a neuronal progenitor marker expressed in LGE and MGE region of forebrain (Wang et al. (2010) J. Neurosci. 30:5334-5345)), OLIG2 (an oligodendrocyte marker), GFAP (a glial marker), DCX (a marker specifying immature neurons), SOX6 (a marker expressed in post-mitotic progenitors of MGE region (Batista-Brito et al. (2009) Neuron 63:466-481)) and GABA (a marker specifically expressed by GABAergic interneurons).

As used herein, expression by a cell of only “low” levels of a biomarker of interest is intended to refer to a level that is at most 20%, and more preferably, less than 20%, less than 15%, less than 10% or less than 5% above background levels (wherein background levels correspond to, for example, the level of expression of a negative control marker that is considered to not be expressed by the cell).

In embodiments, the cells generated by the methods of the disclosure are forebrain neural stem cells (FB-NSCs). As used herein, a “forebrain neural stem cell” or “FB-NSC” refers to a stem cell-derived neural stem cell that expresses at least one biomarker, and preferably two or all three biomarkers, selected from OTX2, FEZF2 and SIX3. An FB-NSC may also express additional biomarkers, including but not limited to beta III-tubulin and/or KI67.

In embodiments, the cells generated by the methods of the disclosure are ventral forebrain neural stem cells (VFB-NSCs), which are more differentiated (more mature) cells than FB-NSCs and committed along the ventral lineage. As used herein, a “ventral forebrain neural stem cell” or “VFB-NSC” refers to a stem cell-derived neural cell that expresses the biomarker NKX2-1. In an embodiment, the VFB-NSC does not express, or only expresses low levels of, the biomarker PAX6. In addition, an VFB NSC may also express additional biomarkers, including but not limited to beta III-tubulin and/or KI67.

In embodiments, the cells generated by the methods of the disclosure are medial ganglionic eminence neural progenitor cells (MGE-NPCs). As used herein, a “medial ganglionic eminence neural progenitor cell” or “MGE-NPC” refers to a stem cell-derived neural cell that expresses at least one biomarker, and preferably two or all three biomarkers, selected from ASCL1, LHX6 and DLX1. An MGE-NPC may also express additional biomarkers, including but not limited to beta III-tubulin and/or KI67.

The committed MGE-NPCs generated by the methods of the disclosure can be further cultured in vitro to generate mature GABAergic interneurons, e.g., according to the culture protocols described herein. As used herein, a mature GABAergic neuron refers to a neuronally-derived cell that expresses the biomarker parvalbumin, and may also express LHX6, MAP2, GABA and/or GAD1.

### II. CULTURE MEDIA COMPONENTS

The methods of the disclosure for generating FB-NSC, VFB-NSC and MGE-NPCs, as well as immatured neurons and parvalbumin+interneurons from the MGE-NPCs, comprise culturing human pluripotent stem cells in culture media comprising specific agonist and/or antagonists of cellular signaling pathways. In various embodiments, the culture media lacks serum, lacks exogenously-added growth factors, lacks animal products, is serum-free, is xeno-free and/or is feeder layer free. In various embodiments, the culture media lacks a SMAD2/3 inhibitor or antagonist, lacks a dual SMAD inhibitor or antagonist or lacks a TGFβ pathway antagonist.

As described in Example 1, a culture media comprising a BMP pathway antagonist, a MEK pathway antagonist, a WNT pathway antagonist, an AKT pathway antagonist, an SHH pathway agonist and a PKC pathway antagonist was sufficient to generate OTX2+FEZF2+SIX3+ forebrain neural stem cells (FB-NSCs) in as little as three days (referred to herein as “stage 1” of the differentiation protocol). Further differentiation of the FB-NSCs in a culture media comprising a BMP pathway antagonist, a MEK pathway antagonist, a WNT pathway antagonist and an SHH pathway agonist was sufficient to generate NKX2-1+VFB-NSCs in another three days (referred to herein as “stage 2”). Still further differentiation of the VFB-NSCs in a culture media comprising a TAK1 pathway antagonist, an SHH pathway agonist, a TGF-β pathway antagonist, a TRK pathway antagonist, a Notch pathway antagonist and an IGF1 pathway agonist was sufficient to generate ASCL1+ medial ganglionic eminence neural progenitor cells (MGE-NPCs) in another three days (referred to herein as “stage 3”), for an overall three-stage nine day protocol for generating MGE-NPCs. The MGE-NPCs can be further differentiated into immatured neurons by further culture for three days in a culture media comprising a CREB or PKA pathway agonist, valproic acid or analog thereof, Substance P or analog thereof, a GDNF pathway agonist and an mTOR pathway agonist (referred to herein as “stage 4”). Finally, the immatured neurons can be further differentiated into mature GAB Aergic interneurons by further culture for two weeks (14 days) in a culture media comprising a BDNF pathway agonist, an IGF-1 pathway agonist, ascorbic acid or analog, sodium pyruvate, O-LPA or analog, N2 supplement and nonessential amino acids (referred to herein as “stage 5”).

As used herein, an “agonist” of a cellular signaling pathway is intended to refer to an agent that stimulates (upregulates) the cellular signaling pathway. Stimulation of the cellular signaling pathway can be initiated extracellularly, for example by use of an agonist that activates a cell surface receptor involved in the signaling pathway (e.g., the agonist can be a receptor ligand). Additionally or alternatively, stimulation of cellular signaling can be initiated intracellularly, for example by use of a small molecule agonist that interacts intracellularly with a component(s) of the signaling pathway.

As used herein, an “antagonist” of a cellular signaling pathway is intended to refer to an agent that inhibits (downregulates) the cellular signaling pathway. Inhibition of the cellular signaling pathway can be initiated extracellularly, for example by use of an antagonist that blocks a cell surface receptor involved in the signaling pathway. Additionally or alternatively, inhibition of cellular signaling can be initiated intracellularly, for example by use of a small molecule antagonist that interacts intracellularly with a component(s) of the signaling pathway.

Agonists and antagonists used in the methods of the disclosure are known in the art and commercially available. They are used in the culture media at a concentration effective to achieve the desired outcome, e.g., generation of forebrain neural stem or progenitor cells expressing forebrain markers of interest. Non-limiting examples of suitable agonist and antagonists agents, and effective concentration ranges, are described further below.

Antagonists of the BMP (bone morphogenetic protein) pathway include agents, molecules, compounds, or substances capable of inhibiting (downregulating) the BMP signaling pathway, which biologically is activated by binding of BMP to a BMP receptor, which are activin receptor-like kinases (ALK) (e.g., type I BMP receptor, including but not limited to ALK2 and ALK3). In one embodiment, the BMP pathway antagonist is selected from the group consisting of LDN193189, DMH1, DMH2, Dorsomorphin, K02288, LDN214117, LDN212854, follistatin, ML347, Noggin, and combinations thereof. In one embodiment, the BMP pathway antagonist is present in the culture media at a concentration within a range of 100-500 nM, 100-400 nM, 150-350 nM or 200-300 nM. In one embodiment, the BMP pathway antagonist is LDN193189. In one embodiment, the BMP pathway antagonist is LDN193189, which is present in the culture media at a concentration within a range of 100-500 nM, 100-400 nM, 150-350 nM or 200-300 nM. In one embodiment, the BMP pathway antagonist is LDN193189, which is present in the culture media at a concentration of 275 nM in step (a) (stage 1) and 250 nM in step (b) (stage 2).

Antagonists of the MEK pathway include agents, molecules, compounds, or substances capable of inhibiting (downregulating) the signaling pathway of one or more of the components of the MAPK/ERK pathway (also known as the Ras-Raf-MEK-ERK pathway). In one embodiment, the MEK pathway antagonist is selected from the group consisting of PD0325901, Binimetinib (MEK162), Cobimetinib (XL518), Selumetinib, Trametinib (GSK1120212), CI-1040 (PD-184352), Refametinib, ARRY-142886 (AZD-6244), PD98059, U0126, BI-847325, RO 5126766, and combinations thereof. In one embodiment, the MEK pathway antagonist is present in the culture media at a concentration within a range of 25-300 nM, 50-150 nM, 50-250 nM, 75-200 nM or 100-120 nM. In one embodiment, the MEK pathway antagonist is PD0325901. In one embodiment, the MEK pathway antagonist is PD0325901, which is present in the culture media at a concentration within a range of 25-300 nM, 50-150 nM, 50-250 nM, 75-200 nM or 100-120 nM. In one embodiment, the MEK pathway antagonist is PD0325901, which is present in the culture media at a concentration of 110 nM in step (a) (stage 1) and 100 nM in step (b) (stage 2).

Antagonists of the WNT pathway include agents, molecules, compounds, or substances capable of inhibiting (downregulating) the canonical Wnt/β-catenin signaling pathway, which biologically is activated by binding of a Wnt-protein ligand to a Frizzled family receptor. In one embodiment, the WNT pathway antagonist is selected from the group consisting of XAV939, ICG001, Capmatinib, endo-IWR-1, IWP-2, IWP-4, MSAB, CCT251545, KY02111, NCB-0846, FH535, LF3, WIKI4, Triptonide, KYA1797K, JW55, JW 67, JW74, Cardionogen 1, NLS-StAx-h, TAK715, PNU 74654, iCRT3, WIF-1, DKK1, and combinations thereof. In one embodiment, the WNT pathway antagonist is present in the culture media at a concentration within a range of 10-500 nM, 50-250 nM or 50-150 nM. In one embodiment, the WNT pathway antagonist is XAV939. In one embodiment, the WNT pathway antagonist is XAV939, which is present in the culture media at a concentration of 10-500 nM, 50-250 nM or 50-150 nM. In one embodiment, the WNT pathway antagonist is XAV939, which is present in the culture media at a concentration of 110 nM in step (a) (stage 1) and 100 nM in step (b) (stage 2).

Agonists of the SHH (sonic hedgehog) pathway include agents, molecules, compounds, or substances capable of stimulating (activating) signaling through the SHH pathway, which biologically involves binding of SHH to the Patched-1 (PTCH1) receptor and transduction through the Smoothened (SMO) transmembrane protein. In one embodiment, the SHH pathway agonist is selected from the group consisting of Purmorphamine, GSA 10, SAG, and combinations thereof. In one embodiment, the SHH pathway agonist is present in the culture media at a concentration within a range of 100-1000 nM, 200-800 nM, 250-750 nM or 500-600 nM. In one embodiment, the SHH pathway agonist is Purmorphamine. In one embodiment, the SHH pathway agonist is Purmorphamine, which is present in the culture media at a concentration of 100-1000 nM, 200-800 nM, 250-750 nM or 500-600 nM. In one embodiment, the SHH pathway agonist is Purmorphamine, which is present in the culture media at a concentration of 550 nM in step (a) (stage 1) and 500 nM in steps (b) and (c) (stages 2 and 3).

Antagonists of the AKT pathway include agents, molecules, compounds, or substances capable of inhibiting (downregulating) the signaling pathway of one or more of the serine/threonine kinase AKT family members, which include AKT1 (also designated PKB or RacPK), AKT2 (also designated PKBβ or RacPK-β) and AKT 3 (also designated PKBγ or thyoma viral proto-oncogene 3). In one embodiment, the AKT pathway antagonist is selected from the group consisting of MK2206, GSK690693, Perifosine (KRX-0401), Ipatasertib (GDC-0068), Capivasertib (AZD5363), PF-04691502, AT 7867, Triciribine (NSC154020), ARQ751, Miransertib (ab235550), Borussertib, Cerisertib, and combinations thereof. In one embodiment, the AKT pathway antagonist is present in the culture media at a concentration within a range of 25-300 nM, 50-200 nM, 75-200 nM or 125-150 nM. In one embodiment, the AKT pathway antagonist is MK2206. In one embodiment, the AKT pathway antagonist is MK2206, which is present in the culture media at a concentration within a range of 25-300 nM, 50-200 nM, 75-200 nM or 125-150 nM. In one embodiment, the AKT pathway antagonist is MK2206, which is present in the culture media at a concentration of 138 nM in step (a) (stage 1).

Antagonists of the PKC (protein kinase C) pathway include agents, molecules, compounds, or substances capable of inhibiting (downregulating) a PKC signaling pathway, which biologically is mediated by a PKC family member. The PKC family of serine/threonine kinases comprises fifteen isozymes, including the “classical” PKC subcategory, which contain the isoforms α, β1, β2 and γ. In one embodiment, the PKC pathway antagonist inhibits the activity of at least one (and in other embodiments, at least two or three) PKC enzyme selected from PKCα, PKCβ1, PKCβ2 and PKCγ. In one embodiment, the PKC pathway antagonist is selected from the group consisting of Go 6983, Sotrastaurin, Enzastaurin, Staurosporine, LY31615, Go 6976, GF 109203X, Ro 31-8220 Mesylate, and combinations thereof. In one embodiment, the PKC pathway antagonist is present in the culture media at a concentration within a range of 10-500 nM, 50-300 nM, 50-150 nM or 75-150 nM. In one embodiment, the PKC pathway antagonist is Go 6983. In one embodiment, the PKC pathway antagonist is Go 6983, which is present in the culture media at a concentration of 10-500 nM, 50-300 nM, 50-150 nM or 75-150 nM. In one embodiment, the PKC pathway antagonist is Go 6983, which is present in the culture media at a concentration of 110 nM in step (a) (stage 1).

Antagonists of the TAK1 (also known as MAP3K7) pathway include agents, molecules, compounds, or substances capable of inhibiting (downregulating) signaling through TAK1 (MAP3K7). In one embodiment, the TAK1 pathway antagonist is selected from the group consisting of Takinib, Dehydoabietic acid, NG25, Sarsasapogenin, and combinations thereof. In one embodiment, the TAK1 pathway antagonist is present in the culture media at a concentration within a range of 1-5 uM, 1-3 uM or 1.5-2.5 uM. In one embodiment, the TAK1 pathway antagonist is Takinib. In one embodiment, the TAK1 pathway antagonist is Takinib, which is present in the culture media at a concentration of 1-5 uM, 1-3 uM or 1.5-2.5 uM. In one embodiment, the TAK1 pathway antagonist is Takinib, which is present in the culture media in step (c) of the method (i.e., stage 3) at a concentration of 2 uM.

Antagonists of the TGFβ (transforming growth factor beta) pathway include agents, molecules, compounds, or substances capable of inhibiting (downregulating) signaling through a TGFβ receptor family member, a family of serine/threonine kinase receptors. In one embodiment, the TGFβ pathway antagonist is selected from the group consisting of A 83-01, SB-431542, GW788388, SB525334, TP0427736, RepSox, SD-208, and combinations thereof. In one embodiment, the TGFβ pathway antagonist is present in the culture media at a concentration within a range of 300-800 nM, 250-750 nM, 300-650 nM or 400-600 nM. In one embodiment, the TGF pathway antagonist is A 83-01. In one embodiment, the TGFβ pathway antagonist is A 83-01, which is present in the culture media at a concentration of 300-800 nM, 250-750 nM, 300-650 nM or 400-600 nM. In one embodiment, the TGFβ pathway antagonist is A 83-01, which is present in the culture media in step (c) of the method (i.e., stage 3) at a concentration of 500 nM.

Antagonists of the TRK pathway include agents, molecules, compounds, or substances capable of inhibiting (downregulating) signaling through TrkA, TrkB and/or TrkC tyrosine kinase. In one embodiment, the TRK pathway antagonist is selected from the group consisting of GNF-5837, BMS-754807, UNC2020, Taletrectinib, Altiratinib, Selitrectinib, PF 06273340, and combinations thereof. In one embodiment, the TRK pathway antagonist is present in the culture media at a concentration within a range of 30-80 nM, 25-75 nM, 30-65 nM or 40-60 nM. In one embodiment, the TRK pathway antagonist is GNF-5837. In one embodiment, the TRK pathway antagonist is A GNF-5837, which is present in the culture media at a concentration of 30-80 nM, 25-75 nM, 30-65 nM or 40-60 nM. In one embodiment, the TRK pathway antagonist is GNF-5837, which is present in the culture media in step (c) of the method (i.e., stage 3) at a concentration of 50 nM.

Antagonists of the Notch pathway include agents, molecules, compounds, or substances capable of inhibiting (downregulating) signaling through or activity of the Notch transcription factor. In one embodiment, the Notch pathway antagonist is selected from the group consisting of GSI-XX, RO4929097, Semagacestat, Dibenzazepine, LY411575, Crenigacestat, IMR-1, IMR-1A, FLI-06, DAPT, Valproic acid, YO-01027, CB-103, Tangeretin, BMS-906024, Avagacestat, Bruceine D, and combinations thereof. In one embodiment, the TRK pathway antagonist is present in the culture media at a concentration within a range of 25-200 nM, 50-150 nM or 75-125 nM. In one embodiment, the Notch pathway antagonist is GSI-XX. In one embodiment, the Notch pathway antagonist is GSI-XX, which is present in the culture media at a concentration of 25-200 nM, 50-150 nM or 75-125 nM. In one embodiment, the Notch pathway antagonist is GSI-XX, which is present in the culture media in step (c) of the method (i.e., stage 3) at a concentration of 100 nM.

Agonists of the IGF1 (insulin-like growth factor 1) pathway include agents, molecules, compounds, or substances capable of stimulating (activating) signaling through the IGF1 pathway. In one embodiment, the IGF1 pathway agonist is selected from the group consisting of IGF1, IGF1-Ado, X10, mecasermin, and combinations thereof. In one embodiment, the IGF1 pathway agonist is present in the culture media at a concentration within a range of 2-20 ng/ml, 5-15 ng/ml or 7.5-12.5 ng/ml. In one embodiment, the IGF1 pathway agonist is IGF1. In one embodiment, the IGF1 pathway agonist is IGF1, which is present in the culture media at a concentration of 2-20 ng/ml, 5-15 ng/ml or 7.5-12.5 ng/ml. In one embodiment, the IGF1 pathway agonist is IGF1, which is present in the culture media at a concentration of 10 ng/ml in step (c) (stage 3) of the method. In one embodiment, the IGF1 pathway agonist is IGF1, which is present in the culture media at a concentration of 10 ng/ml in step (e) (stage 5) of the method.

CREB or PKA pathway agonists include agents, molecules, compounds, or substances capable of stimulating (activating) signaling through the CREB or PKA pathway. In one embodiment, the CREB or PKA pathway agonist is selected from the group consisting of cAMP, Dibutyryl-cAMP, 8-Br-cAMP, cAMPS-Sp, CW 008, Forskolin, 8-CPT-cAMP, CW 008, Adenosine 3′,5′-cyclic Monophosphate, N6-Benzoyl-, Sodium Salt, Adenosine 3′,5′-cyclic monophosphate sodium salt monohydrate, (S)-Adenosine, cyclic 3prime,5prime-(hydrogenphosphorothioate) triethylammonium, Sp-Adenosine 3prime,5prime-cyclic monophosphorothioate triethylammonium salt, Sp-5,6-DCI-cBiMPS, 8-Bromoadenosine 3′,5′-cyclic Monophosphothioate, Sp-Isomer sodium salt, Adenosine 3prime,5prime-cyclic Monophosphorothioate,8-Bromo-, Sp-Isomer, Sodium Salt, Sp-8-pCPT-cyclic GMPS Sodium, 8-Bromoadenosine 3′,5′-cyclic monophosphate, N6-Monobutyryladenosine 3prime:5prime-cyclic monophosphate sodium salt, 8-PIP-cAMP, Sp-cAMPS, and combinations thereof. In one embodiment, the CREB or PKA pathway agonist is present in the culture media at a concentration within a range of 0.5-2.5 μM, 0.75-2.0 μM or 1.0-1.5 μM. In one embodiment, the CREB or PKA pathway agonist is cAMP. In one embodiment, the CREB or PKA pathway agonist is cAMP, which is present in the culture media at a concentration of 0.5-2.5 μM, 0.75-2.0 μM or 1.0-1.5 μM. In one embodiment, the CREB or PKA pathway agonist is cAMP, which is present in the culture media at a concentration of 1.0 μM in stage 4 of the method.

The stage 4 media includes valproic acid or analog thereof. In one embodiment, valproic acid or analog thereof is selected from the group consisting of valproic acid, valproate, sodium valproate and valproate semisodium. In one embodiment, valproic acid or analog is present in the culture media at a concentration within a range of 250-750 nM, 300-600 nM or 450-550 nM. In one embodiment, valproic acid is used. In one embodiment, valproic acid is used at a concentration of 250-750 nM, 300-600 nM or 450-550 nM. In one embodiment, valproic acid is used, which is present in the culture media at a concentration of 500 nM in stage 4 of the method.

The stage 4 media includes Substance P or analog thereof. Substance P is an 11 amino acid neuropeptide, with numerous analogs thereof being described in the art (reviewed in e.g., Datar et al. (2004) Curr. Top. Med. Chem. 4:75-103). In one embodiment, Substance P or analog thereof is present in the culture media at a concentration within a range of 50-250 nM, 75-200 nM or 100-150 nM. In one embodiment, Substance P is used. In one embodiment, Substance P is used at a concentration of 50-250 nM, 75-200 nM or 100-150 nM. In one embodiment, Substance P is used, which is present in the culture media at a concentration of 100 nM in stage 4 of the method.

Agonists of the glial cell line-derived neurotrophic factor (GDNF) pathway include agents, molecules, compounds, or substances capable of stimulating (upregulating) the GDNF signaling pathway. In one embodiment, the GDNF pathway agonist is selected from the group consisting of GDNF, BT13, BT44, and combinations thereof. In one embodiment, the GDNF pathway agonist is present in the culture media at a concentration within a range of 1-100 ng/ml, 5-50 ng/ml, 10-25 ng/ml or 12.5-17.5 ng/ml. In one embodiment, the GDNF pathway agonist is GDNF. In one embodiment, the GDNF pathway agonist is GDNF, which is present in the culture media at a concentration of 1-100 ng/ml, 5-50 ng/ml, 10-25 ng/ml or 12.5-17.5 ng/ml. In one embodiment, the GDNF pathway agonist is GDNF, which is present in the culture media in stage 4 of the method at a concentration of 10 ng/ml.

Agonists of the mTOR (mammalian target of rapamycin) pathway include agents, molecules, compounds, or substances capable of stimulating (upregulating) signaling through mTOR, a PI3K-related kinase family member which is a core component of the mTORC1 and mTORC2 complexes. In one embodiment, the mTOR pathway agonist is selected from the group consisting of MHY1458, NV-5138, Testosterone, 3-benzyl-5-((2-nitrophenoxy) methyl)-dihydrofuran-2(3H)-one (3BDO), 3BDO, L-leucine, NV-5138 hydrochloride, NV-5138, L-leucine-d1, L-leucine-2-13C,15N, Leucine-13C6, L-leucine-d7, L-leucine-d10, L-leucin-d2, 1-leucine-d3, L-leucine-18O2, L-leucine-13C, L-leucine-2-13C, L-leucine-13C6-15N, L-leucine-15N, L-leucine-1-13C,15N, and combinations thereof. In one embodiment, the mTOR pathway agonist is present in the culture media at a concentration within a range of 1-5 μM, 1-3 μM or 1.5-2.5 μM. In one embodiment, the mTOR pathway agonist is MHY1458. In one embodiment, the mTOR pathway agonist is MHY1458, which is present in the culture media at a concentration of 1-5 μM, 1-3 μM or 1.5-2.5 μM. In one embodiment, the mTOR pathway agonist is MHY1458, which is present in the culture media in stage 4 of the method at a concentration of 2 μM.

Agonists of the brain-derived neurotrophic factor (BDNF) pathway include agents, molecules, compounds, or substances capable of stimulating (upregulating) the BDNF signaling pathway. In one embodiment, the BDNF pathway agonist is selected from the group consisting of BDNF, rotigotine, 7,8-DHF, ketamine, tricyclic dimeric peptide-6 (TDP6), LM22A-4, and combinations thereof. In one embodiment, the BDNF pathway agonist is present in the culture media at a concentration within a range of 1-100 ng/ml, 5-50 ng/ml, 10-25 ng/ml or 12.5-17.5 ng/ml. In one embodiment, the BDNF pathway agonist is BDNF. In one embodiment, the BDNF pathway agonist is BDNF, which is present in the culture media at a concentration of 1-100 ng/ml, 5-50 ng/ml, 10-25 ng/ml or 12.5-17.5 ng/ml. In one embodiment, the BDNF pathway agonist is BDNF, which is present in the culture media in stage 5 of the method at a concentration of 10 ng/ml.

The stage 5 media includes ascorbic acid or analog thereof. In one embodiment, ascorbic acid or analog thereof is selected from the group consisting of vitamin C, 2-phospho-L-ascorbic acid, L-ascorbic acid, sodium ascorbyl phosphate, magnesium ascorbyl phosphate, ascorbyl glucoside, tetrahexyldecyl ascorbate (THD), ethylated L-ascorbic acid, and combinations thereof. In one embodiment, ascorbic acid or analog is present in the culture media at a concentration within a range of 50-500 μM, 100-400 μM or 200-300 μM. In one embodiment, 2-phospho-L-ascorbic acid is used. In one embodiment, 2-phospho-L-ascorbic acid is used at a concentration of 50-500 μM, 100-400 μM or 200-300 μM. In one embodiment, 2-phospho-L-ascorbic acid is used, which is present in the culture media at a concentration of 200 μM in stage of the method.

The stage 5 media includes sodium pyruvate or analog thereof. In one embodiment, sodium pyruvate or analog thereof is used at a concentration of 50-500 μM, 100-300 μM or 150-250 μM. In one embodiment, sodium pyruvate is used, which is present in the culture media at a concentration of 200 μM in stage 5 of the method.

The stage 5 media includes lysophosphatidic acid (LPA) or analog thereof. In one embodiment, LPA or analog thereof is selected from the group consisting of lysophosphatidic acid, 2-[[3-(1,3-dioxo-1H-benz[de]isoquinolin-2(3H)-yl)propyl]thio]benzoic acid, 1-Oleoyl lysophosphatidic acid sodium salt, UCM-05194, and combinations thereof. In one embodiment, LPA or analog is present in the culture media at a concentration within a range of 50-500 nM, 100-400 nM or 200-300 nM. In one embodiment, O-LPA is used. In one embodiment, O-LPA is used at a concentration of 50-500 nM, 100-400 nM or 200-300 nM. In one embodiment, 0-LPA is used, which is present in the culture media at a concentration of 200 nM in stage 5 of the method.

The stage 5 media further includes an N2 supplement. As used herein, an “N2 supplement” refers to a mixture of cell culture supplements for promoting the growth of neural-derived cells. Various N2 supplements are known in the art, including but not limited to, N-2 Supplement (ThermoFisher Scientific), N2 Supplement-A (Stem Cell Technologies), N-2 MAX Media Supplement (R&D Systems) and N-2 Supplement (CSH Protocols). An N2 supplement comprises a transferrin family molecule as well as additional growth-promoting molecules. In one embodiment, the N2 supplement comprises holo-transferrin, insulin (recombinant full chain), progesterone, putrescine, selenite, retroprogesterone, medrogestone, norethindrone, chlormadinone acetate, cyproterone acetate, medroxyprogesterone acetate, and megestrol acetate. In one embodiment, the N2 supplement is present in the culture media at a concentration within a range of 0.1%-5%, 0.5%-3% or 1-2% nM. In one embodiment, an N2 supplement is present in the culture media at a concentration of 1% in stage 5 of the method.

The stage 5 media further includes a nonessential amino acid (NEAA) supplement. As used herein, an “NEAA supplement” refers to a mixture of amino acids for promoting cell growth. Various NEAA supplements are known in the art, including but not limited to MEM Non-Essential Amino Acids Solution (ThermoFisher Scientific), OriCell™ NEAA Cell Culture Supplement (Cyagen), and SuperCult® Non-Essential Amino Acid (NEAA) Cell Culture Supplement (Creative Bioarray). In one embodiment, the NEAA supplement comprises alanine, arginine, asparagine, aspartic acid, cysteine, glutamic acid, glutamine, glycine, proline, serine, and tyrosine. In one embodiment, the NEAA supplement is present in the culture media at a concentration within a range of 0.1%-5%, 0.5%-3% or 1-2%. In one embodiment, an NEAA supplement is present in the culture media at a concentration of 1% in stage 5 of the method.

When an agonist or antagonist is used in more than one step of the method, in one embodiment it is the same particular agonist or antagonist that is used for each step in which the agent is present in the culture media. In another embodiment, different agonists or antagonists that affect the same signaling pathway are used in different steps of the method. For example, for the BMP antagonist used in steps (a) and (b) (stages 1 and 2), in one embodiment the same BMP antagonist is used in steps (a) and (b). In another embodiment, different BMP antagonists are used in steps (a) and (b). Similarly, for the IGF-1 pathway agonist used in steps (c) and (e) (stages 3 and 5), in one embodiment the same IGF-1 pathway agonist is used in steps (c) and (e). In another embodiment, different IGF-1 pathway agonists are used in steps (c) and (e).

When an agonist or antagonist is used in more than one step of the method, in one embodiment it is the same concentration of the same agonist or antagonist that is used for each step in which the agent is present in the culture media. In another embodiment, different concentrations of the same agonist or antagonist are used in different steps of the method. For example, for the BMP antagonist used in steps (a) and (b) (stages 1 and 2), in one embodiment the same concentration of the same BMP antagonist is used in steps (a) and (b). In another embodiment, different concentrations of the same BMP antagonist are used in steps (a) and (b). Similarly, for the IGF-1 agonist used in steps (c) and (e) (stages 3 and 5), in one embodiment the same concentration of the same IGF-1 agonist is used in steps (c) and (e). In another embodiment, different concentrations of the same IGF-1 agonist are used in steps (c) and (e).

### III. CULTURE CONDITIONS

In combination with the chemically-defined and optimized culture media described in subsection II above, the methods of generating FB-NSCs, VFB-NSCs, MGE-NPCs, immatured neurons and mature GABAergic interneurons of the disclosure utilize standard culture conditions established in the art for cell culture. For example, cells can be cultured at 37° C. and under 5% O₂ and 5% CO₂ conditions. Cells can be cultured in standard culture vessels or plates, such as 96-well plates. In certain embodiments, the starting pluripotent stem cells are adhered to plates, preferably coated with an extracellular matrix material such as vitronectin. In one embodiment, the stem cells are cultured on a vitronectin coated culture surface (e.g., vitronectin coated 96-well plates).

Pluripotent stem cells can be cultured in commercially-available media prior to differentiation. For example, stem cells can be cultured for at least one day in Essential 8 Flex media (Thermo Fisher #A2858501) prior to the start of the differentiation protocol. In a non-limiting exemplary embodiment, stem cells are passaged onto vitronectin (Thermo Fisher #A14700) coated 96-well plates at 150,000 cells/cm2 density and cultured for one day in Essential 8 Flex media prior to differentiation.

To begin the differentiation protocol, the media the stem cells are being cultured in is changed to a basal differentiation media that has been supplemented with signaling pathway agonists and/or antagonists as described above in subsection II. A basal differentiation media can include, for example, a commercially-available base supplemented with additional standard culture media components needed to maintain cell viability and growth, but lacking serum (the basal differentiation media is a serum-free media) or any other exogenously-added growth factors, such as FGF2, PDGF or HGF. In a non-limiting exemplary embodiment, a basal differentiation media contains 1x IMDM (Thermo Fisher #12440046), 1x F12 (Thermo Fisher #11765047), poly(vinyl alcohol) (Sigma #p8136) at 1 mg/ml, chemically defined lipid concentrate (Thermo Fisher #11905031) at 1%, 1-thioglycerol (Sigma #M6145) at 450 uM, Insulin (Sigma #11376497001) at 0.7 ug/ml and transferrin (Sigma #10652202001) at 15 ug/ml (also referred to herein as “CDM2” media, as used in the exemplary differentiation protocols shown in FIG. 17 and FIG. 18).

The culture media typically is changed regularly to fresh media. For example, in one embodiment, media is changed every 24 hours.

To generate FB-NSCs, VFB-NSCs, MGE-NPCs, immatured neurons and mature GABAergic interneurons, the starting stem cells are cultured in the optimized culture media for sufficient time for cellular differentiation and expression of committed FB-NSC−, VFBNSC−, -MGE-NPC−, immatured neuron- or mature GABAergic interneuron-associated markers. As described in the Examples, it has been discovered that culture of pluripotent stem cells in a five-stage method, one optimized for generation of FB-NSCs, a second optimized for generation of VFB-NSCs, a third optimized for the generation of MGE-NPCs, a fourth optimized for the generation of immatured neurons and a fifth optimized for the generation of mature GAB Aergic interneurons can lead to the production of MGE-NPCs in as little as nine days of culture and mature interneurons in as little as 26 days of culture. The culture period for the first stage (leading to FB-NSCs) is days 0-3, the culture period for the second stage (leading to VFB-NSCs) is days 3-6, the culture period for the third stage (leading to MGE-NPCs) is days 6-9, the culture period for the fourth stage (leading to immatured neurons) is days 9-12 and the culture period for the fifth stage (leading to mature interneurons) is days 12-26.

Accordingly, in the first stage of the method, which generates FB-NSCs, also referred to herein as “step (a)” or “stage 1”, pluripotent stem cells are cultured in the stage 1-optimized culture media on days 0-3, or starting on day 0 and continuing through day 3, or for 72 hours (3 days), or for at least 60 hours, or at least 64 hours, or at least 68 hours, or at least 70 hours, or at least 72 hours, or for 60 hours, or for 64 hours, or for 68 hours, or for 70 hours or for 72 hours.

Accordingly, in the second stage of the method, which generates VFB-NSCs, also referred to herein as “step (b)” or “stage 2”, the FB-NSCs generated in step (a) are further cultured in the stage 2-optimized culture media on days 4-6, or starting on day 4 and continuing through day 6, or starting on day 4 and continuing for 72 hours (3 days), or starting on day 4 and continuing for at least 60 hours, or at least 64 hours, or at least 68 hours, or at least 70 hours, or at least 72 hours, or starting on day 4 and continuing for 60 hours, or for 64 hours, or for 68 hours, or for 70 hours or for 72 hours.

Accordingly, in the third stage of the method, which generates MGE-NPCs, also referred to herein as “step (c)” or “stage 3”, the VFB-NSCs generated in step (b) are further cultured in the stage 3-optimized culture media on days 6-9, or starting on day 6 and continuing through day 9, or starting on day 6 and continuing for 72 hours (3 days), or starting on day 6 and continuing for at least 60 hours, or at least 64 hours, or at least 68 hours, or at least 70 hours, or at least 72 hours, or starting on day 6 and continuing for 60 hours, or for 64 hours, or for 68 hours, or for 70 hours or for 72 hours.

Accordingly, in the fourth stage of the method, which generates immatured neurons, also referred to herein as “step (d)” or “stage 4”, the MGE-NPCs generated in step (c) are further cultured in the stage 4-optimized culture media on days 9-12, or starting on day 9 and continuing through day 12, or starting on day 9 and continuing for 72 hours (3 days), or starting on day 9 and continuing for at least 60 hours, or at least 64 hours, or at least 68 hours, or at least 70 hours, or at least 72 hours, or starting on day 9 and continuing for 60 hours, or for 64 hours, or for 68 hours, or for 70 hours or for 72 hours.

Accordingly, in the fifth stage of the method, which generates mature GABAergic interneurons, also referred to herein as “step (e)” or “stage 5”, the immatured neurons generated in step (d) are further cultured in the stage 5-optimized culture media on days 12-26, or starting on day 12 and continuing through day 26, or starting on day 12 and continuing in culture for sufficient time to generate parvalbumin+mature interneurons (e.g., 14 days, or two weeks, of culture in the stage 5 media).

### IV. USES

The methods and compositions of the disclosure for generating FB-NSCs, VFB-NSCs, MGE-NPCs, immatured neurons and mature GABAergic interneurons allow for efficient and robust availability of these cell populations for a variety of uses. For example, the methods and compositions can be used in the study of forebrain neural progenitor development and biology, including differentiation into GAB Aergic interneurons, to assist in the understanding and potential treatment of neurodegenerative and psychiatric diseases and disorders involving dysfunction of GABAergic interneurons. For example, FB-NSCs, VFB-NSCs, MBE-NPCs, immatured neurons and mature GABAergic interneurons generated using the methods of the disclosure can be further purified according to methods established in the art using agents that bind to surface markers expressed on the cells. Accordingly, in one embodiment, the disclosure provides a method of isolating FB-NSCs, VFB-NSCs, MGE-NPCs, immatured neurons or mature GABAergic interneurons, the method comprising:
contacting FB-NSCs, VFB-NSCs, MGE-NPCs, immatured neurons or mature GABAertic interneurons generated by a method of the disclosure with at least one binding agent that binds to a cell surface marker expressed by the FB-NSCs, VFB-NSCs, MGE-NPCs, immatured neurons or mature GABAergic interneurons; and
isolating cells that bind to the binding agent to thereby isolate the FB-NSCs, VFB-NSCs, MBE-NPCs, immatured neurons or mature GABAergic interneurons.

In one embodiment, the binding agent is an antibody, e.g., a monoclonal antibody (mAb) that binds to the cell surface marker. Cells that bind the antibody can be isolated by methods known in the art, including but not limited to fluorescent activated cell-sorting (FACS) and magnetic activated cell sorting (MACS).

Progenitors of the forebrain neural lineage also are contemplated for use in the treatment of neurodegenerative or psychiatric diseases and disorders, through delivery of the cells to a subject having the disease or disorder, including any such disease or disorder involving dysfunction of GABAergic interneurons. Transplantation of embryonic medial ganglionic eminence (MGE) cells into the adult brain has been shown to result in dispersal and migration of the transplanted cells and differentiation into neurons expressing GABA (Alvarez-Dolado et al. (2006) J. Neurosci. 26:7380-7389). Accordingly, in one embodiment, forebrain neural lineage progenitors are used in the treatment of epilepsy. Transplantation of precursors of GABAergic interneurons into the postnatal neocortex of mice has been shown to reduce epileptic seizures (Baraban et al. (2009) Proc. Natl. Acad. Sci. USA 106:15472-15477). The use of neural progenitors in the treatment of epilepsy is also reviewed in Shetty and Upadhya (2016) Neurosci. Biobehav. Rev. 62:35-47 and Lybrand et al. (2020) Neuropharm. 168:107781.

Deficiency in, or reduced activity of, GAB Aergic interneurons also is associated with the cognitive deficits of Alzheimer's disease or Autism patients. Accordingly, the forebrain lineage cells of the disclosure also can be used in the treatment of disorders associated with cognitive impairment that may benefit from restoration of GABAergic interneuron function, including but not limited to Alzheimer's disease and Autism.

The cells of the disclosure also are useful in the screening of potential drugs or for the development of novel cell therapies for the treatment of diseases or disorders involving dysfunction of GAB Aergic interneurons.

### V. COMPOSITIONS

In other aspects, the disclosure provides compositions related to the methods of generating FB-NSCs, VFB-NSCs, MGE-NPCs, immatured neurons and mature GABAergic interneurons, including culture media and cell cultures, as well as isolated progenitor cells and cell populations.

In one aspect, the disclosure provides a culture media for obtaining human OTX2+FEZF2+SIX3+ forebrain neural stem cells (FB-NSCs) comprising a BMP pathway antagonist, a MEK pathway antagonist, a WNT pathway antagonist, an AKT pathway antagonist, an SHH pathway agonist and a PKC pathway antagonist. Suitable agents, and concentrations therefor, include those described in subsection II.

In one aspect, the disclosure provides a culture media for obtaining human NKX2-1+ ventral forebrain neural stem cells (VFB-NSCs) comprising a BMP pathway antagonist, a MEK pathway antagonist, a WNT pathway antagonist and an SHH pathway agonist. Suitable agents, and concentrations therefor, include those described in subsection II.

In one aspect, the disclosure provides a culture media for obtaining human ASCL1+ medial ganglionic eminence neural progenitor cells (MGE-NPCs) comprising a TAK1 pathway antagonist, an SHH pathway agonist, a TGF-β pathway antagonist, a TRK pathway antagonist, a Notch pathway antagonist and an IGF1 pathway agonist. Suitable agents, and concentrations therefor, include those described in subsection II.

In one aspect, the disclosure provides a culture media for obtaining human immatured neurons from MGE-NPCs, the media comprising a CREB or PKA pathway agonist, valproic acid or analog, Substance P or analog, a GDNF pathway agonist and an mTOR pathway agonist. Suitable agents, and concentrations therefor, include those described in subsection II.

In one aspect, the disclosure provides a culture media for obtaining human mature GABAergic interneurons, the media comprising a BDNF pathway agonist, an IGF-1 pathway agonist, ascorbic acid or analog, sodium pyruvate or analog, LPA or analog, an N2 supplement and an NEAA supplement. Suitable agents, and concentrations therefor, include those described in subsection II.

In one aspect, the disclosure provides an isolated cell culture of human OTX2+FEZF2+SIX3+ forebrain neural stem cells (FB-NSCs), the culture comprising human OTX2+FEZF2+SIX3+FB-NPCs cultured in a culture media comprising a BMP pathway antagonist, a MEK pathway antagonist, a WNT pathway antagonist, an AKT pathway antagonist, an SHH pathway agonist and a PKC pathway antagonist. Suitable agents, and concentrations therefor, include those described in subsection II.

In one aspect, the disclosure provides an isolated cell culture of human NKX2-1+ ventral forebrain neural stem cells (VFB-NSCs), the culture comprising human NKX2-1+VFB-NPCs cultured in a culture media comprising a BMP pathway antagonist, a MEK pathway antagonist, a WNT pathway antagonist and an SHH pathway agonist. Suitable agents, and concentrations therefor, include those described in subsection II.

In one aspect, the disclosure provides an isolated cell culture of human ASCL1+ medial ganglionic eminence neural progenitor cells (MGE-NPCs), the culture comprising human ASCL1+MGE-NPCs cultured in a culture media comprising a TAK1 pathway antagonist, an SHH pathway agonist, a TGF-β pathway antagonist, a TRK pathway antagonist, a Notch pathway antagonist and an IGF1 pathway agonist. Suitable agents, and concentrations therefor, include those described in subsection II.

In one aspect, the disclosure provides an isolated cell culture of human immatured neurons, the culture comprising human immatured neurons cultured in a culture media comprising a CREB or PKA pathway agonist, valproic acid or analog, Substance P or analog, a GDNF pathway agonist and an mTOR pathway agonist. Suitable agents, and concentrations therefor, include those described in subsection II.

In one aspect, the disclosure provides an isolated cell culture of human mature GABAergic interneurons, the culture comprising human mature GABAergic interneurons cultured in a culture media comprising a BDNF pathway agonist, an IGF-1 pathway agonist, ascorbic acid or analog, sodium pyruvate or analog, LPA or analog, an N2 supplement and an NEAA supplement. Suitable agents, and concentrations therefor, include those described in subsection II.

In one aspect, the disclosure provides human OTX2+FEZF2+SIX3+ forebrain neural stem cells (FB-NSCs) generated by a method of the disclosure (i.e., step (a) or stage 1 of the culture protocol).

In one aspect, the disclosure provides human NKX2-1+ ventral forebrain neural stem cells (VFB-NSCs) generated by a method of the disclosure (i.e., steps (a) and (b), or stages 1 and 2, of the culture protocol).

In one aspect, the disclosure provides human ASCL1+ medial ganglionic eminence neural progenitor cells (MGE-NPCs) generated by a method of the disclosure (i.e., steps (a), (b) and (c), or stages 1, 2 and 3, of the culture protocol).

In one aspect, the disclosure provides human immatured neurons generated by a method of the disclosure (i.e., steps (a), (b), (c), and (d) or stages 1, 2, 3, and 4 of the culture protocol).

In one aspect, the disclosure provides human matured parvalbumin+GABAergic interneurons generated by a method of the disclosure (i.e., steps (a), (b), (c), (d) and (e) or stages 1, 2, 3, 4 and 5 of the culture protocol).

The present invention is further illustrated by the following examples, which should not be construed as further limiting. The contents of figures and all references, patents and published patent applications cited throughout this application are expressly incorporated herein by reference.

### EXAMPLES

### Example 1: Protocol Development for the Generation of Stem Cell Derived Medial Ganglionic Eminence Neural Progenitors Expressing NKX2-1 and ASCL1

A three-stage recipe for generation of medial ganglionic eminence-derived neural progenitors was developed that can guide human pluripotent stem cells to progenitors expressing NKX2-1 and ASCL1 after 9 days in culture. These cells can be further differentiated to mature GABAergic interneurons.

This example utilizes a method of High-Dimensional Design of Experiments (HD-DoE), as previously described in Bukys et al. (2020) Iscience 23:101346. The method employs computerized design geometries to simultaneously test multiple process inputs and offers mathematical modeling of a deep effector/response space. The method allows for finding combinatorial signaling inputs that control a complex process, such as during cell differentiation. It allows testing of multiple plausible critical process parameters, as such parameters impact output responses, such as gene expression. Because gene expression provides hallmark features of the phenotype of, for example, a human cell, the method can be applied to identify, and understand, which signaling pathways control cell fate. In the current example, the HD-DOE method was applied with the intent to find conditions for induction of midbrain neural progenitor-expressed genes, directly from the pluripotent stem cell state.

To develop the recipe for each stage, the impact of agonists and antagonists of multiple signaling pathways (herein called effectors) on the expression of two sets of 53 pre-selected genes, after a 3-day treatment, has been tested and modeled. These effectors are small molecules or proteins that are commonly used during stepwise differentiation of stem cells to specific fates. Choice of the effectors were based on current literature on neural induction in forebrain region of developing brain and differentiation of stem cells to neural progenitors.

To test the effectors, experiments with at least 8 factors were designed that can assess the response of cells to 48 or more different combinations of effectors in a range of concentrations. To analyze the models, we focused on expression of genes involved in development and patterning of the anterior neuroectoderm and forebrain including OTX2 (Hoch et al. (2015) Cell Reports 12:482-494), SIX3 (Lagutin et al. (2003) Genes & Development 17:368-379) and FEZF2 (Zhang et al. (2014) Development 141:4794-47805). At later stages we focused on dorsoventral patterning of telencephalon and gene expressed in ventral forebrain including NKX2-1 and absence of PAX6 which is a dorsal forebrain marker (Stoykova et al. (2000) J. Neurosci. 20:8042-8050). The impact of each effector on gene expression level is defined by a parameter called factor contribution that is calculated for each effector during the modeling.

### Stage 1 Differentiation to Forebrain-Committed Neural Stem Cells

To identify the recipe for stage 1 of differentiation, 96 different combinations of effectors generated using Design-of-Experiments compression through D-optimality were robotically prepared. The effector combinations were prepared in a basal media and were subsequently added to the cells, which were then allowed to differentiate. Three days later RNA extraction was performed, and gene expression was obtained using quantitative PCR analysis. The data were normalized and modeled using partial least squares regression analysis to the effector design, resulting in the generation of gene-specific models, which after model tuning for maximal Q2 predictive power, provided explanation of the effectors ability to control the expression of individual genes, combinatorially, and individually. Solutions within the tested space could then be explored to address desirability.

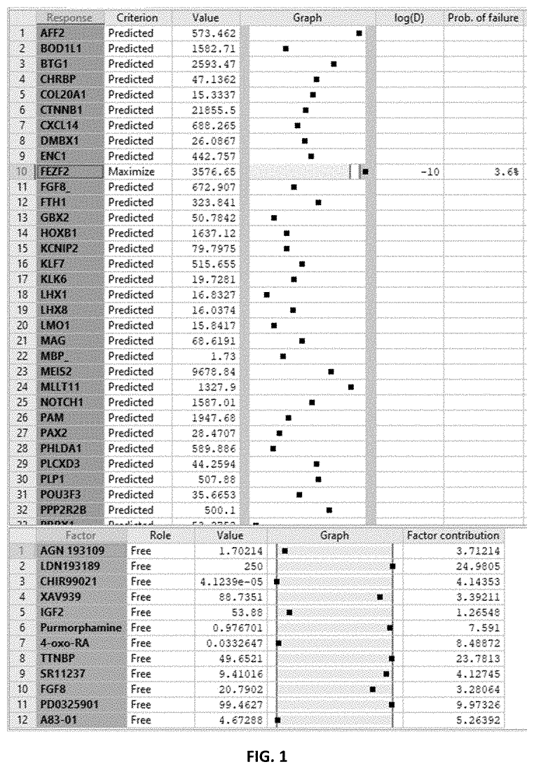

Optimizing for maximal expression of SIX3 at 3300 and FEZF2 1355 expression values led to robust solutions. This model was derived from testing 12 effectors including AGN193109, LDN193189, CHIR99021, XAV939, IGF2, Purmorphamine, 4-oxo-RA, TTNPB, SR11237, FGF8, PD0325901 and A8301. As shown in FIG. 1, three factors, LDN193189 which is an inhibitor of BMP signaling pathway, XAV939 which is an inhibitor of WNT signaling pathway, and Purmorphamine, which is an agonist of SHH pathway, had positive impact on expression of SIX3 with 16, 5 and 5 factor contributions, respectively. This model also showed TTNPB which is a RA agonist, had a significant negative effect on expression of SIX3 with factor contribution of 21. Within the specifications of attaining 80% maximal expression of SIX3, this complex media composition had a Cpk value (process capability index) of 0.6, with a corresponding to a 3.4% risk of failure.

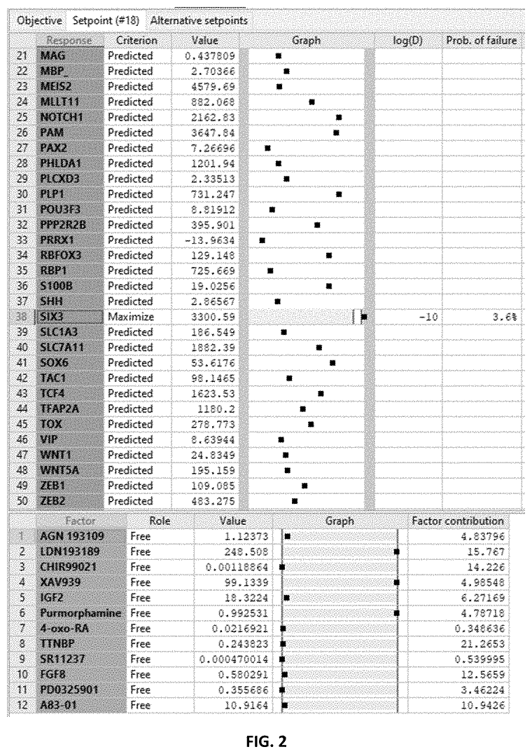

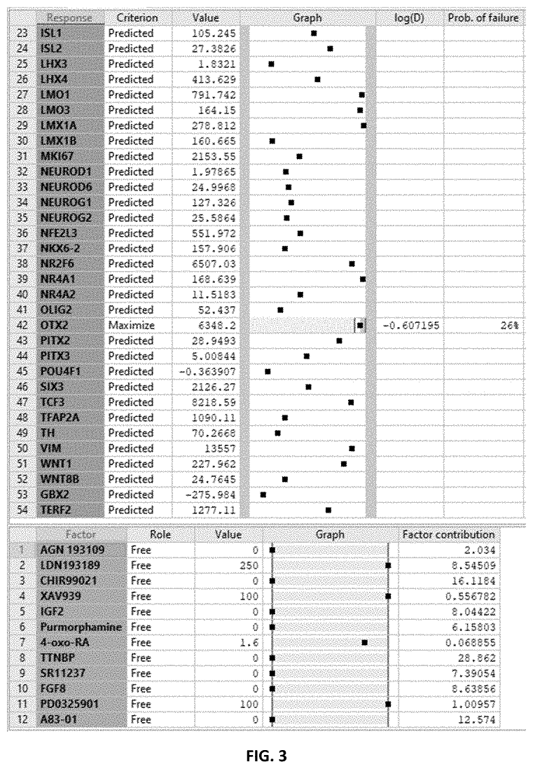

The model was also optimized for maximum expression of FEZF2 separately at 3576 expression value to find any additional factors that can boost expression of forebrain genes. Within the specifications of attaining 80% maximal expression of FEZF2, this complex media composition had a Cpk value (process capability index) of 0.61, with a corresponding to a 3.3% risk of failure. As shown in FIG. 2, LDN193189 again was identified with a significant positive impact on expression of FEZF2 with factor contribution of 25; PD0325901 which is a MEK inhibitor, TTNPB and Purmorphamine with factor contributions of 10, 24 and 7 also had positive impact on its expression. Maximum expression of OTX2 at 6348 was also modeled and within the specifications of attaining 80% maximal expression of OTX2, this complex media composition had a Cpk value (process capability index) of 0.49, with a corresponding to a 6.6% risk of failure. As shown in FIG. 3, the model showed LDN193189 as the highest positive effector with factor contribution of 8. XAV939 and PD0325901 also showed positive impact, however their factor contributions were low. The effector with highest factor contribution was TTNPB that had a significant negative impact with factor contribution of 29.

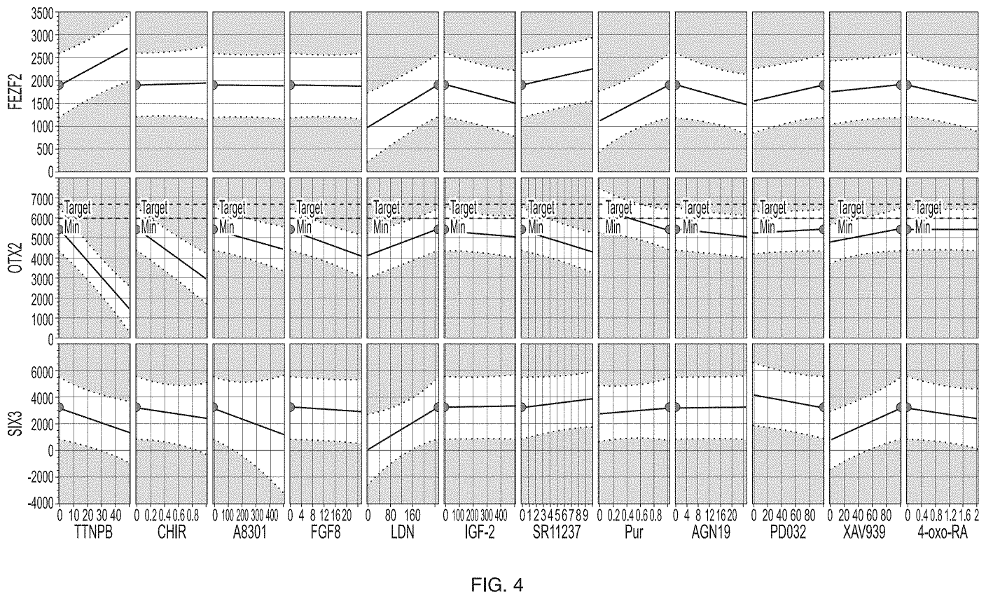

To find the set of factors that have the potential to optimize all 3 genes in this experiment, a new assessment was done through dynamic profile analysis with focus on maximal expression of OTX2, FEZF2 and SIX3. The results are shown in FIG. 4. Based on this analysis, TTNPB that was shown to have negative impact on both OTX2 and SIX3, was removed from the recipe; and Purmorphamine that had a negative effect on OTX2 but positive impact on SIX3 and FEZF2, was included.

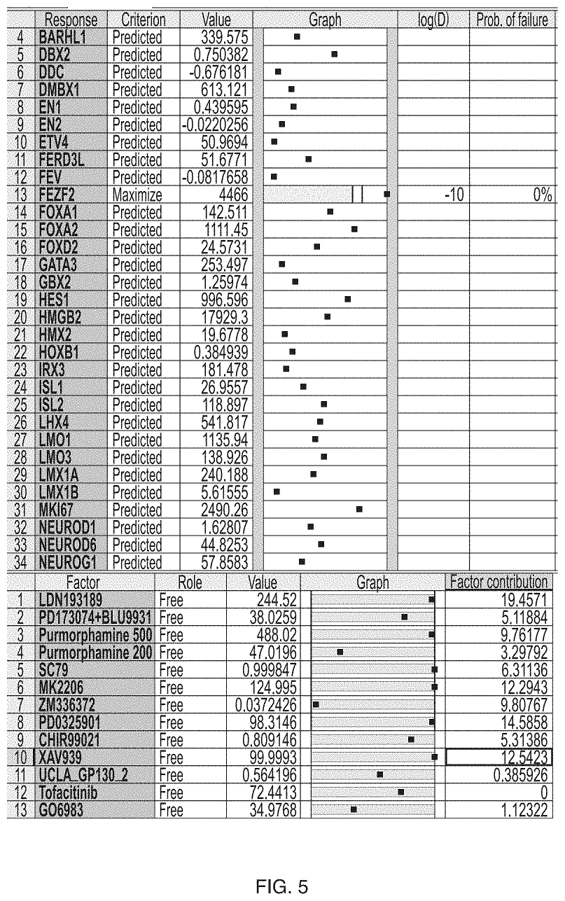

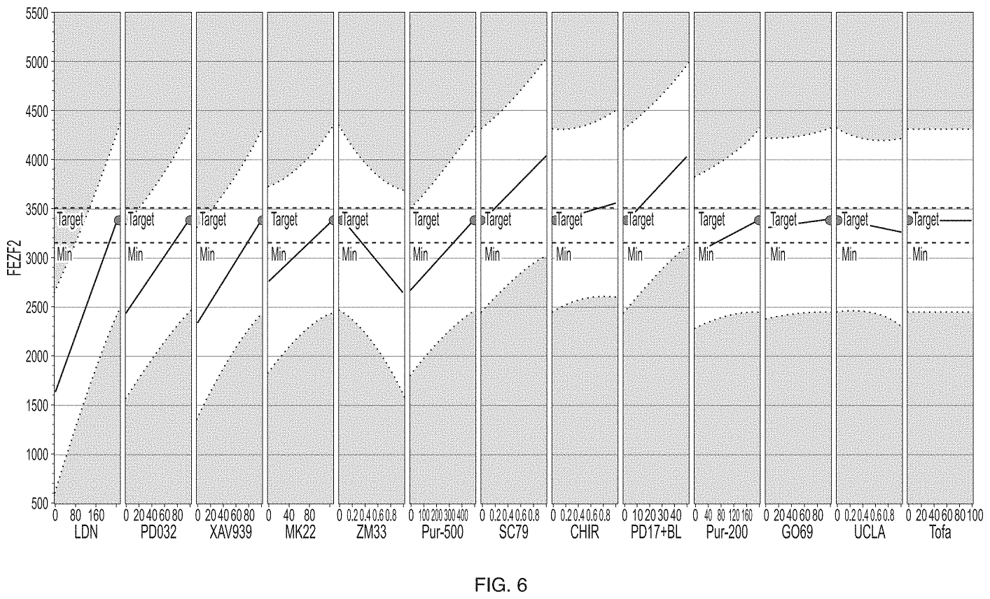

To further enhance the conditions for forebrain differentiation from pluripotency, an additional HD-DoE experiment was performed. Additional gene regulatory models were obtained that were used for preparation of the differentiation protocol. The factors in this experiment included LDN193189, PD173074, BLU9931, Purmorphamine, SC79, MK2206, ZM336372, PD0325901, CHIR99021, XAV939, UCLA-GP130, Tofacitinib and GO6983. As shown in FIG. 5, when optimized for FEZF2, LDN193189, Purmorphamine, MK2206 which is an AKT inhibitor, PD0325901 and XAV939 had positive impact on its expression with factor contributions of 18, 11, 11, 15 and 12, respectively. Within the specifications of attaining 80% maximal expression of FEZF2, this complex media composition had a Cpk value (process capability index) of 0.63, with a corresponding to a 2.7% risk of failure. Dynamic profile analysis was also used to assess the interaction between the effectors. As shown in FIG. 6, the factors that would bring the expression level of FEZF2 outside of target range were eliminated and the remaining effectors included LDN193189, PD0325901, XAV939, Purmorphamine, MK2206 and GO6983.

Considering both HD-DoE experiments, conditions that maximize differentiation of cells to the forebrain region with neural stem cell identity as such relate to robust and elevated expression of FEZF2, SIX3 and OTX2 included the following effector inputs: LDN193189, PD0325901, XAV939, Purmorphamine, MK2206 and GO6983. A representative recipe for Stage 1 differentiation is summarized below in Table 1.

TABLE 1
Validated Effectors for Stage 1 Recipe

| Effectors | Role | concentration |
|---|---|---|
| LDN193189 | BMP inhibitor | 275 nM |
| PD0325901 | MEK inhibitor | 110 nM |
| XAV939 | WNT inhibitor | 110 nM |
| MK2206 | AKT inhibitor | 138 nM |
| Purmorphamine | SHH agonist | 550 nM |
| GO6983 | PKC inhibitor | 110 nM |

### Stage 2 Differentiation to Ventral Forebrain Neural Stem Cells

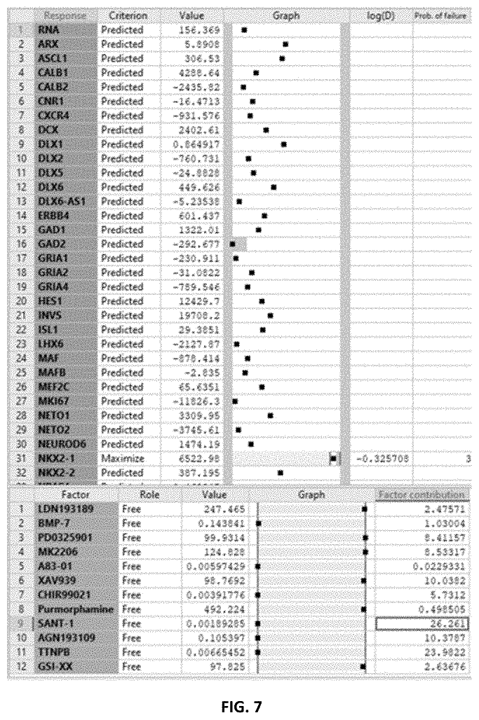

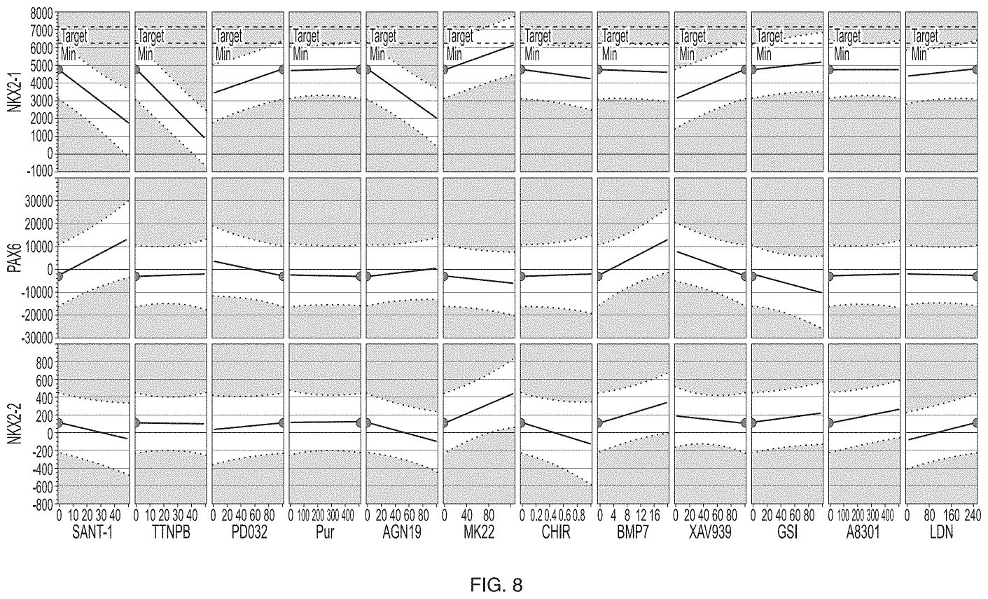

To further guide the differentiation of forebrain-committed neural stem cells to ventral forebrain neural stem cells at stage 2, a HD-DoE experiment was performed for 3 days after termination of stage 1 treatment. At this time, we focused on maximum expression of NKX2-1 that is expressed in ventral forebrain region and minimum expression of PAX6 that is expressed in dorsal forebrain region. This 12-factor experiment included LDN193189, BMP7, PD0325901, MK2206, A8301, XAV939, CHIR99021, Purmorphamine, SANT-1, AGN193109, TTNPB and GSI-XX. As shown in FIG. 7, when the model was maximized for expression of NKX2-1, six effectors were identified that could increase its expression level including LDN193189, PD0325901, MK2206, XAV939, Purmorphamine and GSI-XX which is an inhibitor of Notch signaling pathway. Using dynamic profile analysis, the model was modified to achieve the minimum expression level of PAX6 and NKX2-2 simultaneously. As shown in FIG. 8, this resulted in elimination of MK2206 and GSI-XX.

Considering the model at different optimization settings for maximum differentiation of cells toward ventral forebrain, the recipe for stage 2 of differentiation included LDN193189, PD0325901, XAV939 and Purmorphamine. A representative recipe for Stage 2 differentiation is summarized below in Table 2.

TABLE 2
Validated Effectors for Stage 2 Recipe

| Effectors | Role | concentration |
|---|---|---|
| LDN193189 | BMP inhibitor | 250 nM |
| PD0325901 | MEK inhibitor | 100 nM |
| XAV939 | WNT inhibitor | 100 nM |
| Purmorphamine | SHH agonist | 500 nM |

### Stage 3 Differentiation to Medial Ganglionic Eminence Progenitors

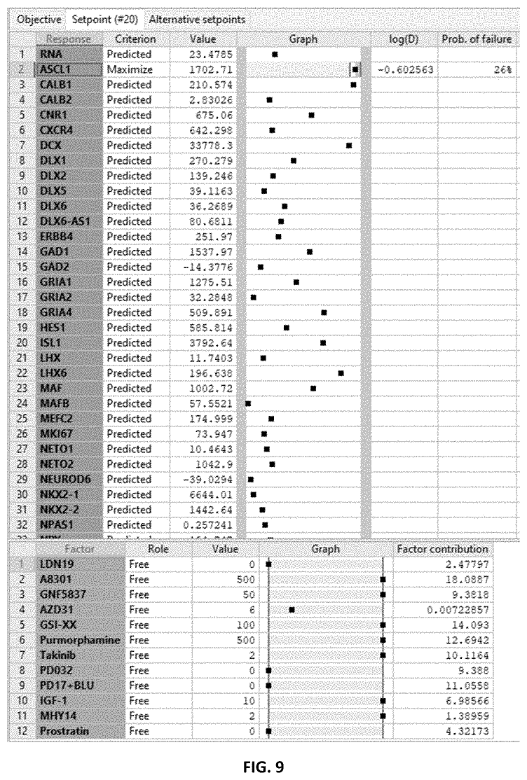

To further guide the cells to medial ganglionic eminence progenitor fate, an additional HD-DoE experiment was performed for 3 days on cells that were treated with stage 1 and stage 2 media for total of 6 days prior to start of the experiment. This experiment included 13 effectors, LDN193189, A8301, GNF5837, AZD3147, GSI-XX, Takinib, PD0325901, PD173074, BLU9931, IGF-1, MHY1485, Purmorphamine and Prostratin. In this model, we focused on maximum expression of genes expressed in neuronal progenitor cells in medial ganglionic eminence (MGE) region including ASCL1, LHX6 and DLX1 (Silberberg et al. (2016) Neuron 92:59-74). When the model was optimized for maximum expression of ASCL1 at 1700, A8301, GSI-XX, Purmorphamine and Takinib, which is an inhibitor of TAK1 pathway, had the highest factor contributions, 18.1, 14.1, 12.7 and 10.1 respectively. GNF5837 and IGF-1 also had positive impact on its expression with factor contributions of 9.4 and 7. MHY1485 also had positive contribution however the factor was below 2 (FIG. 9). Within the specifications of attaining 80% maximal expression of ASCL1, this complex media composition had a Cpk value (process capability index) of 0.59, with a corresponding to a 3.6% risk of failure.

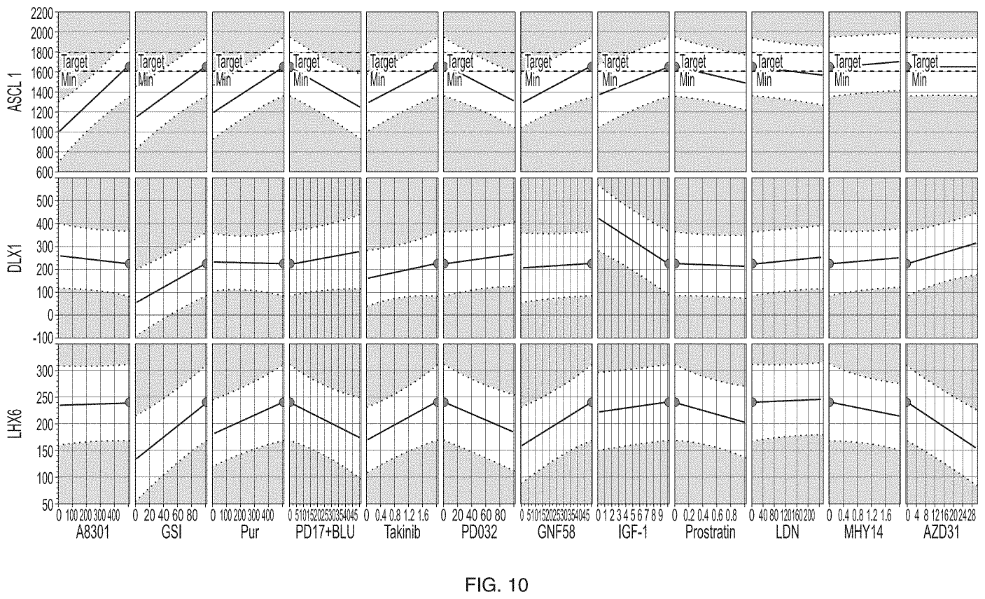

Dynamic profile analysis was used to find common factors with positive impact on expression of ASCL1, LHX6 and DLX1. As shown in FIG. 10, it was observed GSI-XX, Takinib and GNF5837 had similar effect on expression of all three genes and while A8301 and Purmorphamine showed positive trends only for ASCL1 and LHX6, they did not have a significant negative impact on DLX1. Despite the negative impact of IGF-1 on expression level of DLX1, because of its positive effect on ASCL1 and LHX6, it was added to the recipe. Therefore, six effectors including A8301, GSI-XX, Takinib, GNF5837, IGF-1 and Purmorphamine were finalized as ingredients of stage 3 differentiation media. A representative recipe for Stage 3 differentiation is summarized below in Table 3.

TABLE 3
Validated Effectors for Stage 3 Recipe

| Effectors | Role | concentration | |
|---|---|---|---|
| Takinib | Tak inhibitor | 2 | uM |
| Purmorphamine | SHH agonist | 500 | nM |
| A8301 | TGF-b inhibitor | 500 | nM |
| GNF5837 | Trk inhibitor | 50 | nM |
| GSI-XX | Notch inhibitor | 100 | nM |
| IGF-1 | Growth factor | 10 | ng/ml |

### Example 2: Factor Criticality Analysis of Stem Cell Derived Ventral Forebrain Neural Progenitor-Inducing Culture Conditions

To assess the impact of the elimination of each validated factor, dynamic profile analysis was used and compared the expression level of genes of interest in absence of each finalized factor while others are present. Since expression levels of genes of interest reveal whether the desired outcome is reachable, this factor criticality analysis revealed the extent of importance of each input effector.

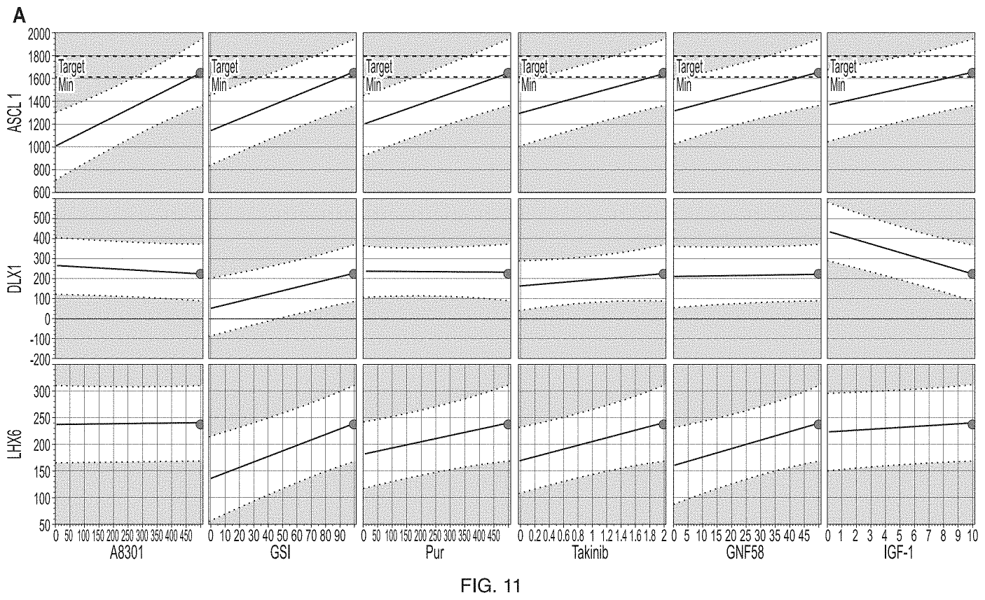

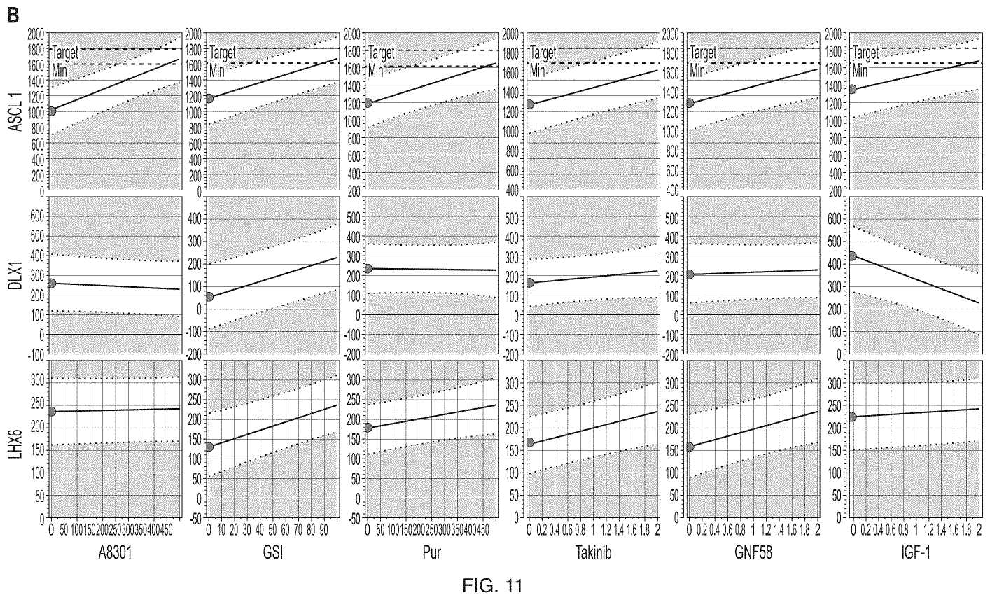

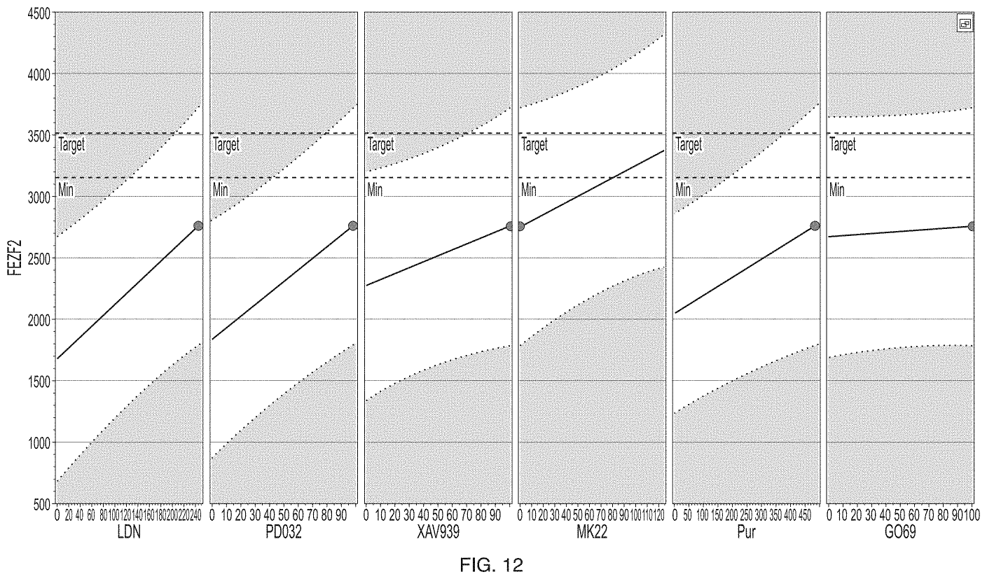

In the stage 1 recipe, each of the six finalized factors were removed individually while the other five factors were present and expression levels of forebrain genes was assessed compared to the presence of all six factors together. The results are summarized in FIG. 11A-B. When LDN193189 was removed, expression levels of FEZF2, OTX2 and SIX3 were decreased drastically from 2000 to 1000, 5500 to 4000 and 3000 to 0, respectively. Absence of XAV939 had a similar effect and levels of FEZF2, OTX2 and SIX3 dropped to 1700, 5000 and 1000, respectively. When Purmorphamine was removed, levels of FEZF2 and SIX3 were decreased to 1000 and below 3000, while levels of OTX2 increased to 6500. Removal of PD0325901 resulted in a drop in levels of FEZF2 but it did not have a significant impact on the other two genes. The decrease of expression levels of FEZF2 from 3500 to 2700 and 3200, as shown in FIG. 12.

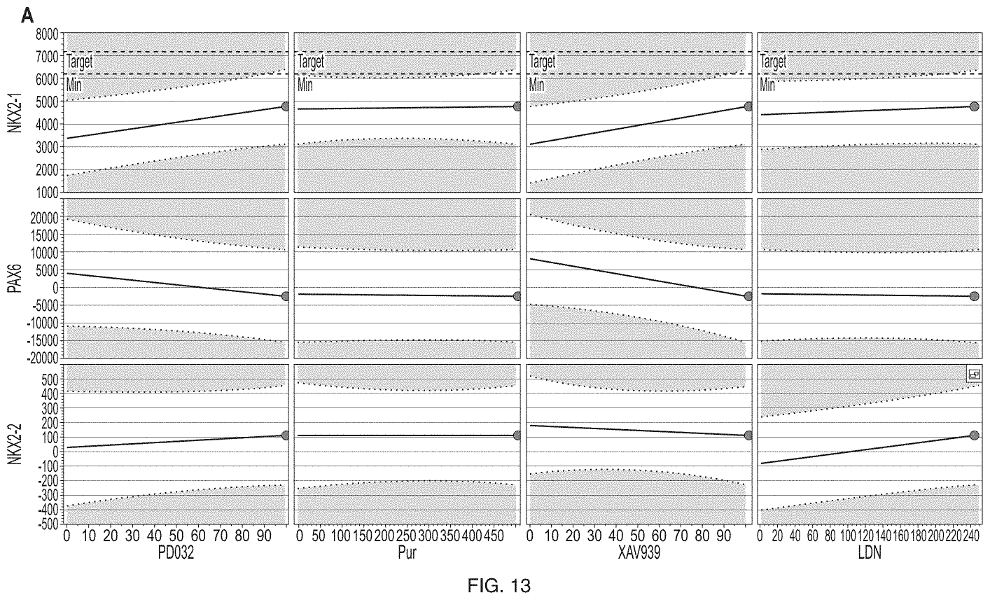

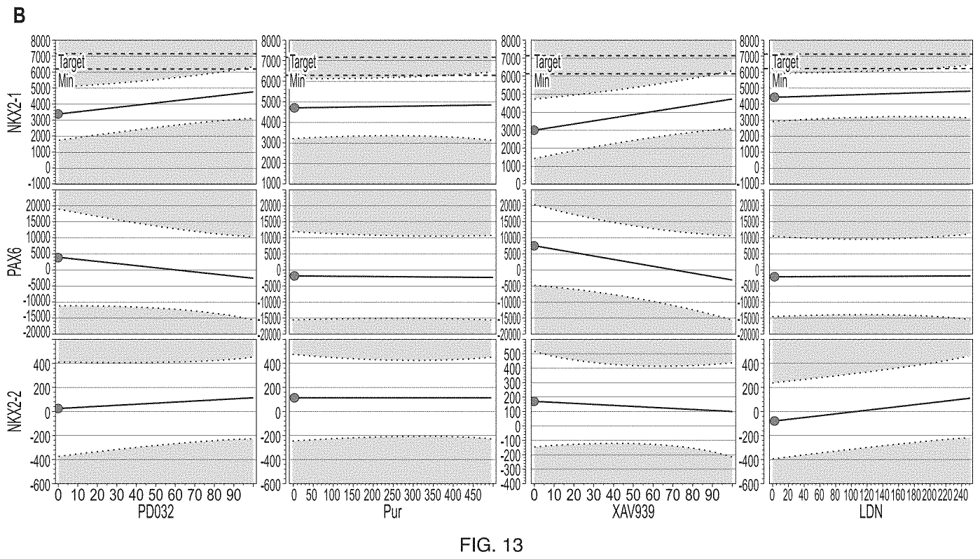

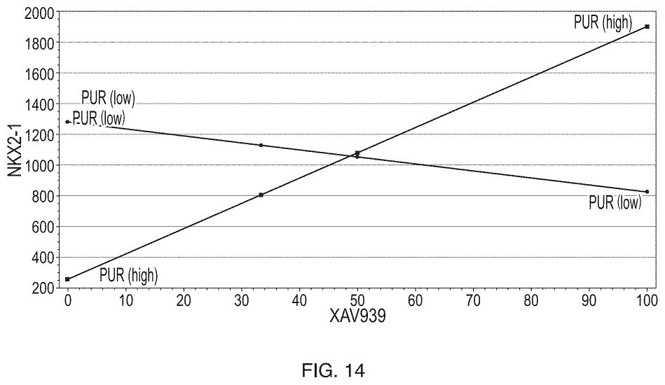

In the stage 2 recipe, each of the four finalized factors were removed while the other three factors remained present and expression levels of NKX2-1, PAX6 and NKX2-2 were assessed compared to presence of all four factors. The results are summarized in FIG. 13A-B. Absence of PD0325901 reduced the levels of NKX2-1 and NKX2-2 while increasing PAX6 from 5000 to 3200, 100 to 0 and 0 to 5000, respectively. In the absence of LDN193189, levels of NKX2-1 did not significantly change, however, levels of both PAX6 and NKX2-2 reduced to below 0. Removing XAV939 reduced the levels of NKX2-1 from 5000 to 3000, while increasing both PAX6 and NKX2-2 to 10000 and 200, respectively. Another observation was the interaction between XAV939 and Purmorphamine. In the absence of XAV939, the impact of Purmorphamine on NKX2-1 became negative and, as it is shown in interaction plot of FIG. 14, the level of NKX2-1 is higher when both factors are present.

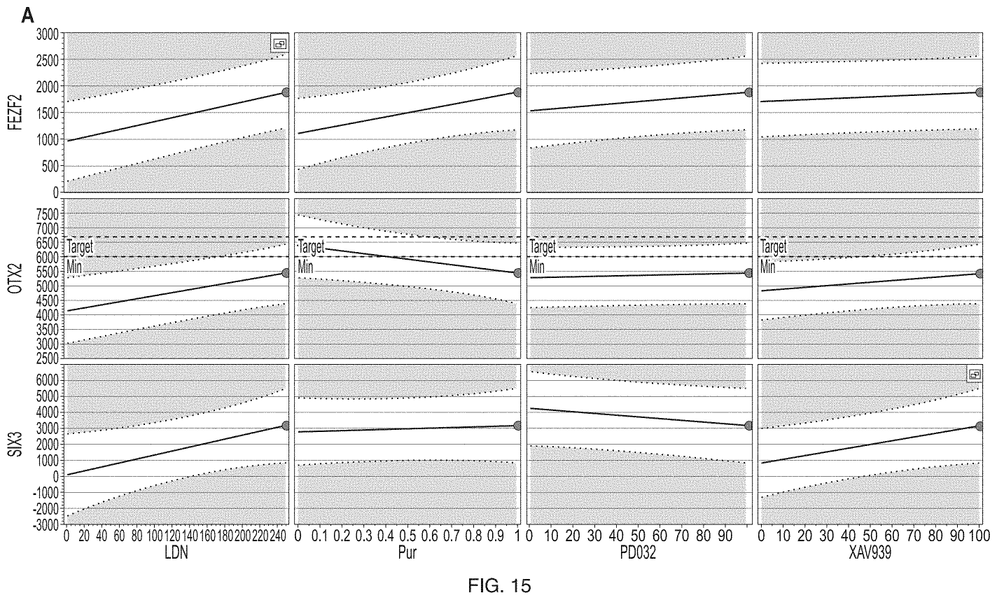

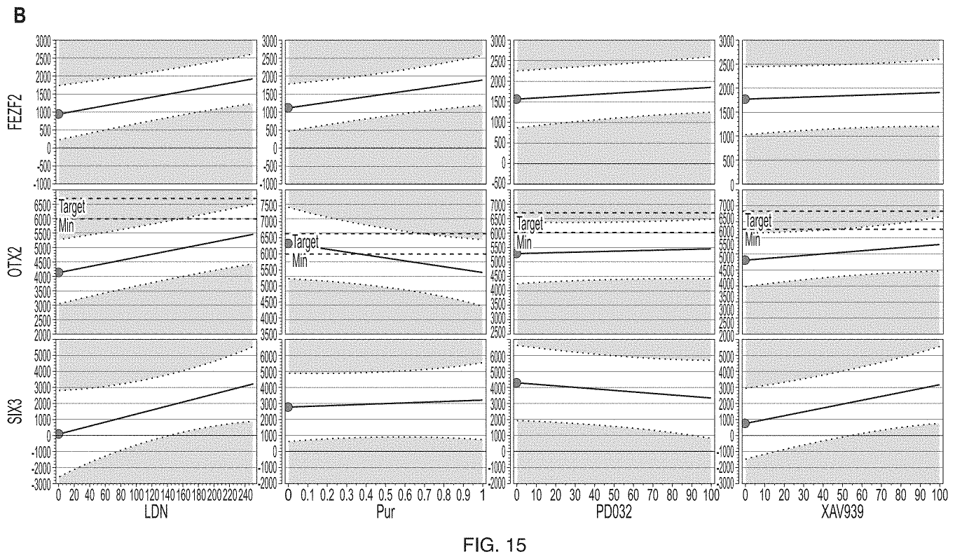

In the stage 3 recipe, each of the six finalized factors were removed while the other five factors remained present and the expression levels of ASCL1, DLX1 and LHX6 were assessed compared to the presence of all six factors. The results are summarized in FIG. 15A-B. By removing A8301, the level of ASCL1 decreased from 1650 to 1000, however values of DLX1 and LHX6 did not change significantly. Removing GSI-XX had a significant impact on the values of all three genes and decreased the expression levels of ASCL1, DLX1 and LHX6 from 1650, 220 and 240 to 1200, 50 and 140, respectively. Removing Takinib had a similar effect on ASCL1, however, expression levels of DLX1 and LHX6 dropped to 180 and 170. In the absence of Purmorphamine, the values of ASCL1 and LHX6 dropped to 1200 and 190 but DLX1 stayed the same. Similar to Purmorphamine, removing GNF5837 resulted in lower expression of ASCL1 at 1300 and LHX6 at 160 but did not have a significant impact on DLX1. As expected, in the absence of IGF-1, the value of DLX1 increases to 410, however, ASCL1 and LHX6 decreased to 1390 and 220, respectively.

### Example 3: Immunocytochemistry Validation of Stem Cell Derived Ventral Forebrain Neural Progenitors Expressing NKX2-1 and ASCL1

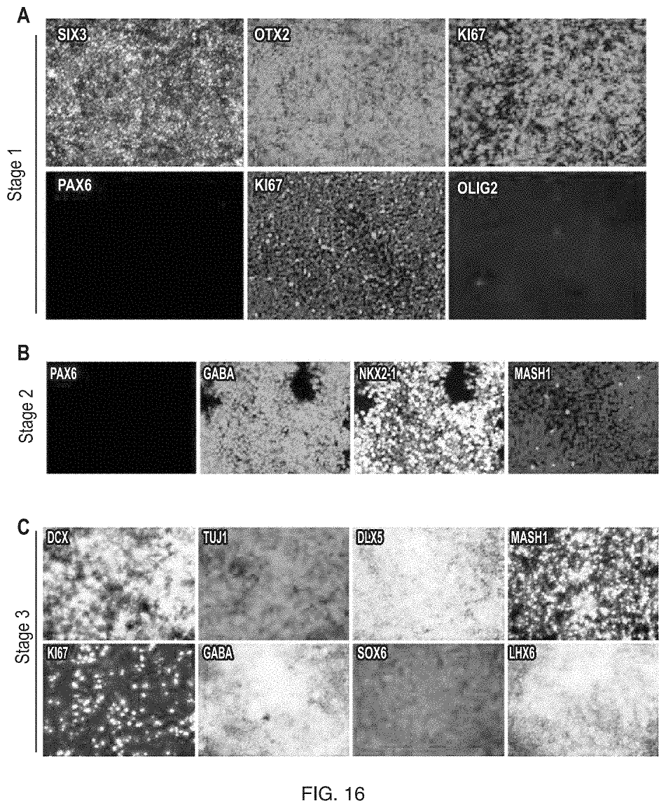

To validate the developed recipes described in example 1, cells were treated with stage 1, stage 2 and stage 3 differentiation media, and immunocytochemistry was used to assess biomarkers of ventral forebrain region and neural progenitors at the end of each stage. Biomarkers included SIX3, OTX2, beta III-tubulin a pan neuronal marker, NKX2-1, PAX6, KI67 a proliferation marker, DLX5 a neuronal progenitor marker expressed in LGE and MGE region of forebrain (Wang et al. (2010) J. Neurosci. 30:5334-5345), LHX6, OLIG2 an oligodendrocyte marker, MASH1 (ASCL1), GFAP a glial marker, DCX a marker specifying immature neurons, SOX6 a marker expressed in post-mitotic progenitors of MGE region (Batista-Brito et al. (2009) Neuron 63:466-481) and GABA a marker specifically expressed by GABAergic interneurons. The results are shown in FIG. 16, FIG. 17 and FIG. 18.

Immunocytochemistry images confirmed the expression of SIX3 and OTX2 by the end of stage 1 in more than 90% of the cells and PAX6 and OLIG2 were not detected. KI67 was also detected in most of the cells, which confirmed cells are proliferative at this point (FIG. 16). After treatment with the stage 2 media, expression of NKX2-1 and GABA was observed in most cells of the culture, with a few of them also expressing MASH1, which confirmed the ventral regionalization of differentiating cells. while PAX6 stayed undetected (FIG. 17). By day 9, which is the end of stage 3, NKX2-1, DLX5 and MASH1 were detected in most of the cells in culture and beta III-tubulin, DCX, LHX6, and SOX6 were detected in more than half the culture that confirmed the commitment of cells to MGE region of forebrain (FIG. 18). KI67 was also detected in some of the cells in culture, which showed these cells still have the ability to proliferate and expand and therefore the recipe results in a mixed culture of proliferative and post mitotic MGE-committed cells.

The detection of ventral forebrain neural progenitor markers and the absence of dorsal forebrain markers in differentiated cells after 9 days confirmed the efficiency and robustness of the generated recipes for a 3-stage differentiation protocol for human induced pluripotent stem cell-derived MGE-committed neural progenitors.

### Example 4: Protocol Development for MGE-Neural Progenitor-Derived Neurons Expressing Parvalbumin and GABA

In this example, two-stage recipes for the neuron maturation from the medial ganglionic eminence (MGE)-derived neural progenitors were developed that can guide MGE progenitor cells to matured PVALB interneurons expressing Parvalbumin and GABA after 17 days in culture.

To develop the recipe for each stage, the impact of agonists and antagonists of multiple signaling pathways, here called effectors, on expression of two sets of 53 pre-selected genes after a 3-day treatment, has been evaluated and modeled. These effectors are small molecules or proteins. They are commonly used during stepwise differentiation of stem cells to specific fates. The choice of the effectors was based on current literature on neural induction in forebrain region of developing brain and differentiation of stem cells to neural progenitors.

The effectors in the final stage 4 and stage 5 recipes developed herein are shown below in Tables 4 and 5, respectively.

TABLE 4
Validated effectors in stage 4 recipe

| Effectors | Roles | Concentration | |
|---|---|---|---|
| cAMP | CREB or PKA | 1 | uM |
| | pathway agonist | | |
| Valproic acid | Valproic acid or | 500 | nM |
| | analog | | |
| Substance P | Substance P or | 100 | nM |
| | analog | | |
| GDNF | GDNF agonist | 10 | ng/mL |
| MHY1458 | mTOR agonist | 2 | uM |

TABLE 5
Validated effectors in stage 5 recipe

| Effectors | Roles | Concentration | |
|---|---|---|---|
| BDNF | BDNF pathway agonist | 10 | ng/mL |
| IGF-1 | IGFR pathway agonist | 10 | ng/mL |
| 2-phospho-L- | Ascorbic acid or analog | 200 | uM |
| ascorbic acid | | | |
| Sodium pyruvate | Sodium pyruvate or analog | 100 | uM |
| O-LPA | LPA or analog | 200 | nM |
| N2 | N2 Supplement | 1% | |
| NEAA | NEAA Supplement | 100 | uM |

To evaluate the effectors, experiments with at least 8 factors were designed that can assess the response of cells to 48 or more different combinations of effectors in a range of concentrations. To analyze the models, we focused on expression of genes involved in development and patterning of the forebrain interneurons maturation including LHX6 (Yuan et al. (2018) Elife, 7:e37382), DLX5 (Wang et al. (2010) J. Neuroscience 30:5334-5345), ASCL1 (Shi et al. (2016) J. Biol. Chem. 291:13560-13570). At later stages we focused on interneuron maturation genes including Parvalbumin and absence of somatostatin (Horn and Nicoll (2018) Proc. Natl. Acad. Sci. 115:589-594). The impacts of each effector on gene expression level are defined by a parameter called factor contribution that is calculated for each effector during the modeling.

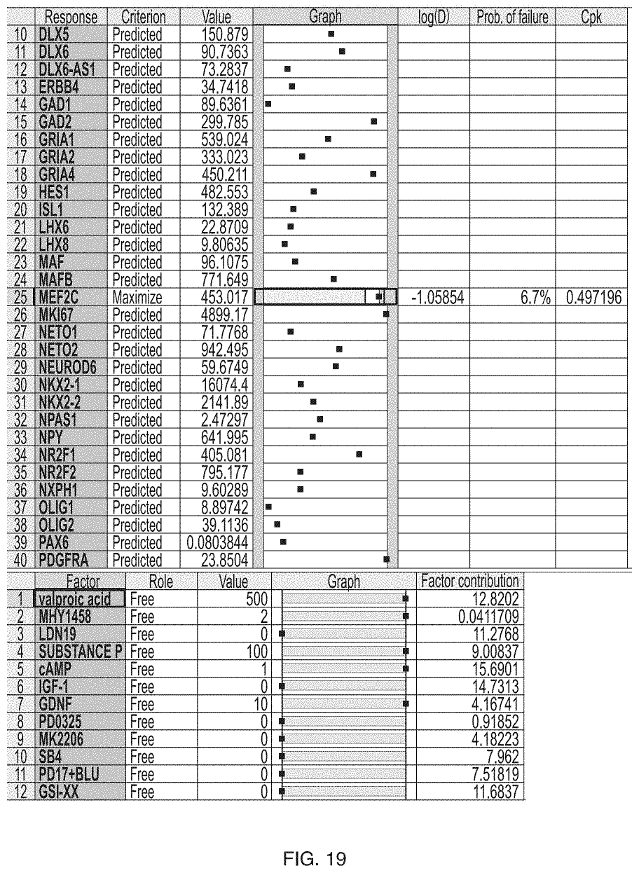

To identify the recipe of stage 4 of differentiation, 48 different combinations of effectors generated using Design-of-Experiments compression through D-optimality were robotically prepared. The effector combinations were prepared in a basal media and were subsequently added to the cells, which were then allowed to differentiate. Three days later RNA extraction was performed, and gene expression was obtained using quantitative PCR analysis. The data was normalized and modeled using partial least squares regression analysis to the effector design, resulting in the generation of gene-specific models, which after model tuning for maximal predictive power, provided explanation of the effectors ability to control the expression of individual genes, combinatorically, and individually. Solutions within the tested space could then be explored to address desirability. Optimizing for maximal expression of MEF2C (Pai et al. (2020) Elife 9:54903) at 453 values led to robust solutions. This model was derived from testing 12 effectors including cAMP, IGF-1, Valproic Acid, GSI-XX, LDN193189, Substance P, SB431542, PD173074+BLU-554, MK2206, GDNF, PD0325901 and MHY1458. Valproic Acid can inhibit histone deaceylases and increase γ-aminobutyric acid (GABA). MHY1458 which is an activator of mTOR signaling pathway, Substance P which is a member of the tachykinin neuropeptide family and cAMP is the activator of CREB and PKA pathway. GDNF is the ligand of Glial cell line-derived neurotrophic factor and can activate the GDNF pathway. They had positive impact on expression of MEF2C with 12, 0.04, 9, 15, and 5 factor contributions, respectively (FIG. 19). This model also showed LDN193189 which is a BMP inhibitor, SB431542 that is TGFβR inhibitor, PD17/BLU which is FGFR inhibitor, GSI-XX which is Notch inhibitor, had a significant negative effect on expression of MEF2C with factor contribution of 11, 7.9, 7.5, 11.6. Within the specifications of attaining 80% maximal expression of MEF2C, this complex media composition had a Cpk value (process capability index) of 0.4, with a corresponding to a 6.7% risk of failure.

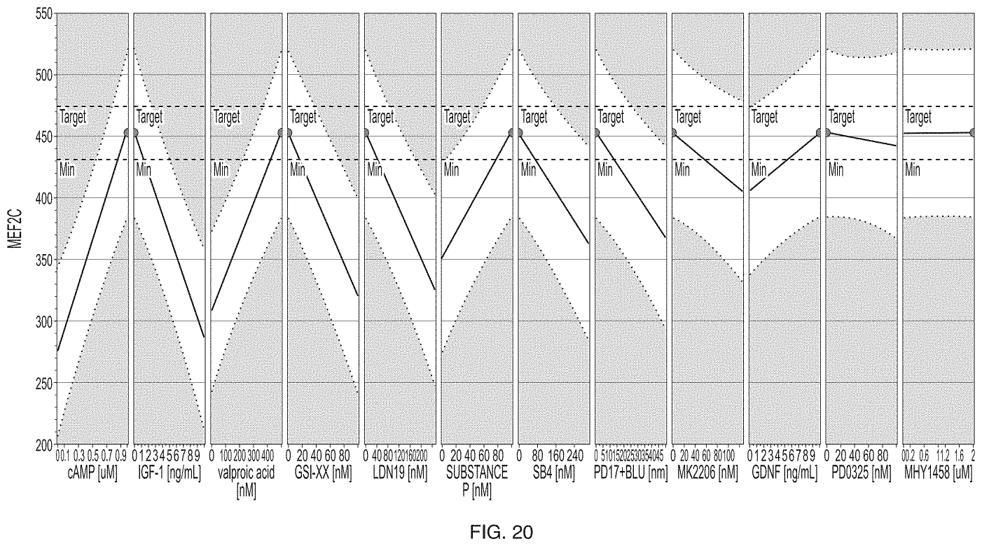

To find the set of factors that have the potential to optimize MEF2C in this experiment, a new assessment was done through dynamic profile analysis with focus on maximal expression of MEF2C (FIG. 20). Based on this analysis, IGF-1 that was shown to have negative impact on MEF2C, was removed from the recipe; and GSI-XX, LDN19, SB431542, and PD17 that had a negative effect on MEF2C, they were also removed.

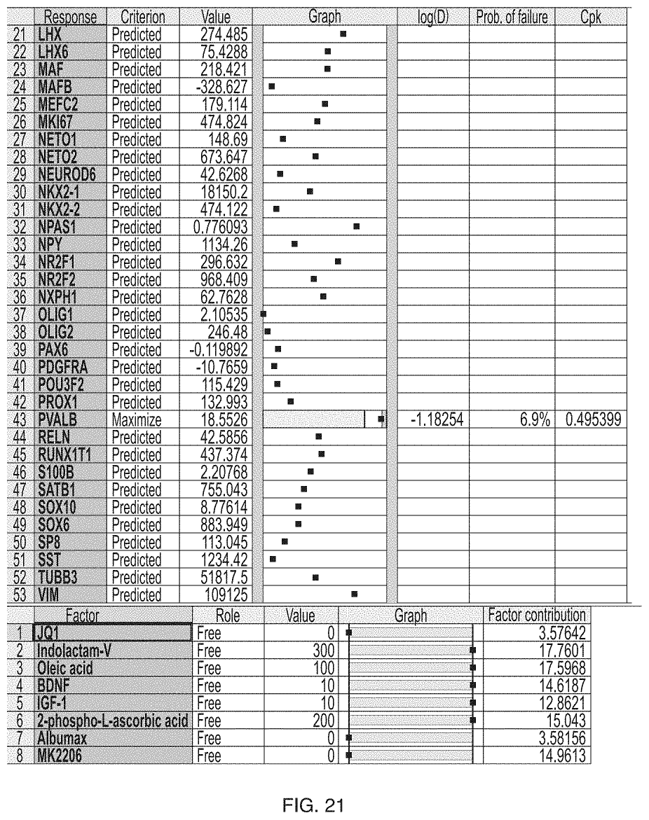

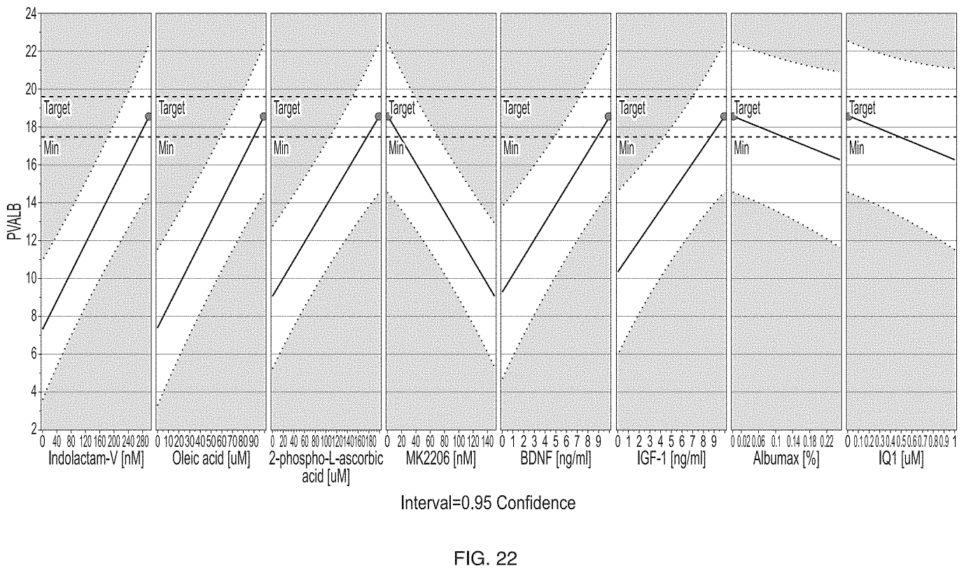

To further guide the differentiation of forebrain-committed neural stem cells to PVALB interneuron cells at stage 5, we performed a HD-DoE experiment for 3 days after termination of stage 4 treatment. At this time, we focused on maximum expression of PVALB that was expressed in Parvalbumin (+) interneurons and minimum expression of Somatostatin that is another type of interneurons. This 8-factor experiment included JQ1, Indolactam-V, Oleic acid, BDNF, IGF-1, 2-phospho-L-Ascorbic acid, Albumax, MK2206. When the model was maximized for expression of PVALB, we identified five effectors that could increase its expression level including Indolactam-V, Oleic acid, BDNF, IGF-1, 2-phospho-L-ascorbic acid, BDNF that is ligand of BDNFR signaling pathway (FIG. 21). Using dynamic profile analysis, we modified the model to achieve the maximum expression level of PVALB simultaneously and therefore 2-phospho-L-ascorbic acid, BDNF, IGF1 were included while MK2206, Albumax, and JQ1 are excluded (FIG. 22). Considering the model at different optimization settings for maximum differentiation of cells toward ventral forebrain, the recipe for stage 5 of differentiation included BDNF, IGF-1, 2-phospho-L-ascorbic acid from this HD-Doe experiment.

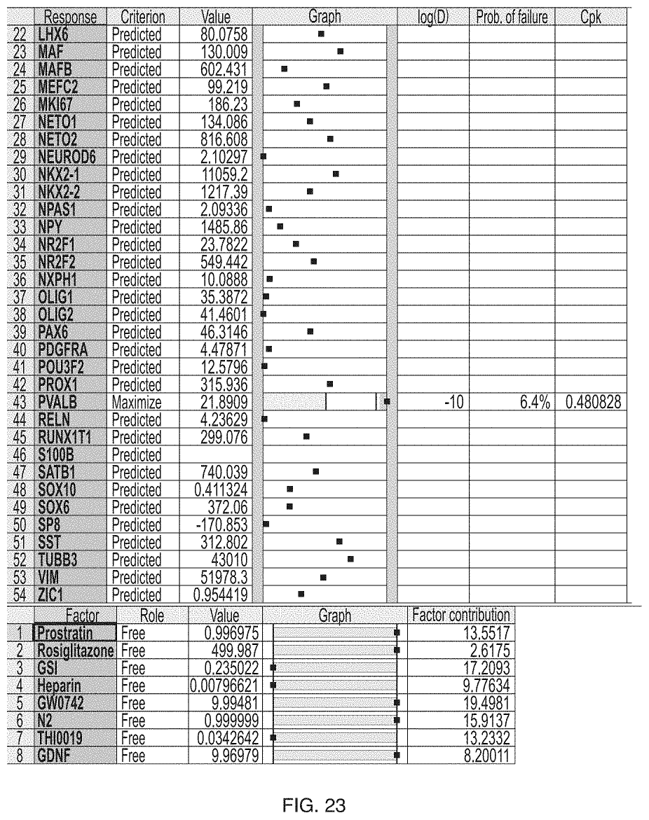

To investigate effect of other factors on differentiation of forebrain-committed neural stem cells to PVALB interneuron cells, an additional HD-DoE experiment was performed for 3 days on cells that were treated with stage 1 to stage 4 media for total of 12 days prior to start of the experiment. This experiment included eight effectors, Prostratin, Rosiglitazone, GSI-XX, Heparin, GW0742, N2, THI0019, and GDNF. In this model, we focused on maximum expression of PVALB gene expressed in PVALB (+) interneurons (Nahar et al. (2021) Front. Psych. 12:679960). When model was optimized for maximum expression of PVALB, Prostratin, GW0742, N2, and GDNF had the highest factor contributions, 13.56, 19.499, 15.9 and 8.2 respectively (FIG. 23). The contribution of N2 has a significant effect on PVALB gene expression, so it is included in our stage 5 recipe.

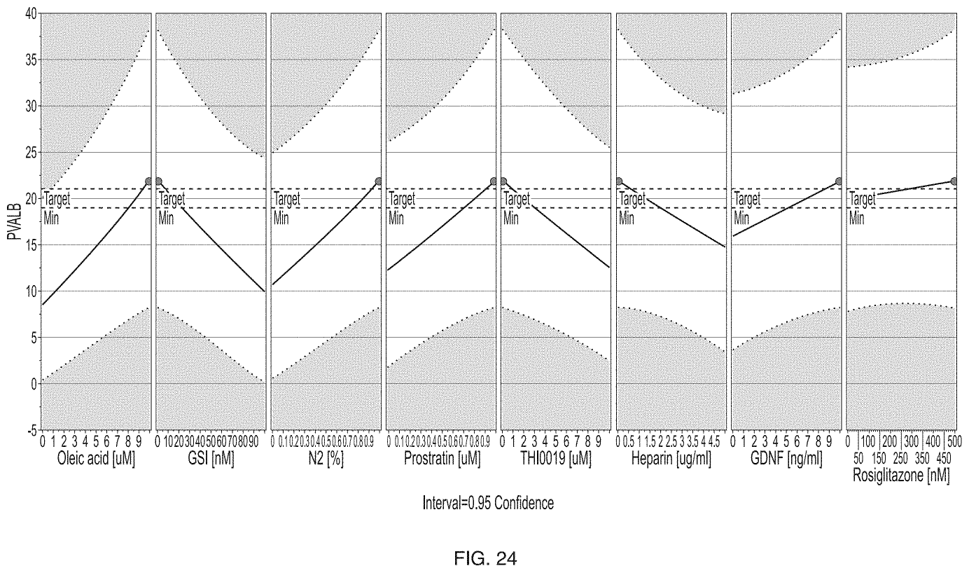

Dynamic profile analysis was used to find common factors with positive impact on expression of PVALB (FIG. 24). It was observed GW0742, N2, Prostratin, and GDNF had similar effect on expression of PVALB gene and GSI-XX, THI0019, Heparin had a negative impact on PVALB gene expression. So, they were excluded in the recipe.

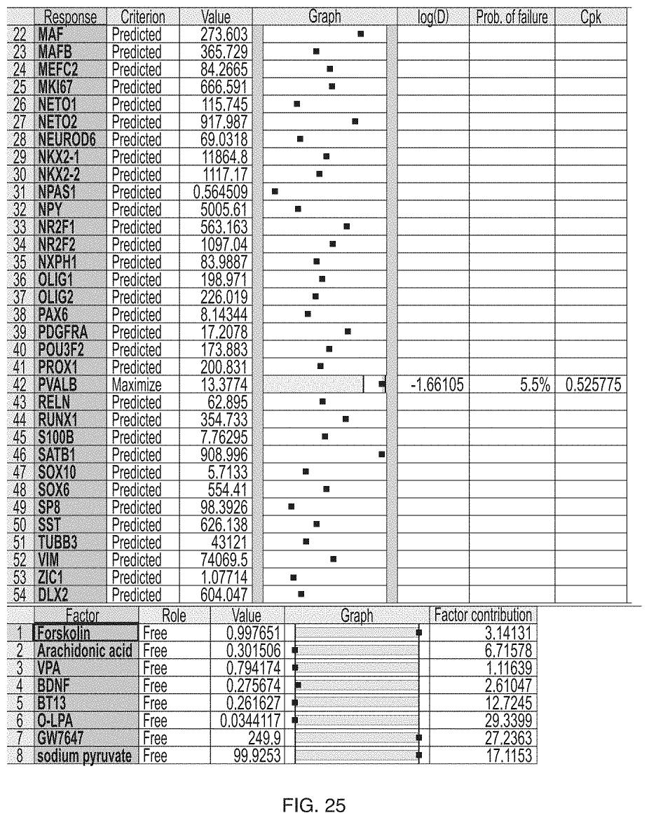

To further guide the differentiation of forebrain-committed neural stem cells to PVALB interneuron cells, an additional HD-DoE experiment was performed for 3 days on cells that were treated with stage 1 to stage 4 media for total of 12 days prior to start of the experiment. This experiment included 8 effectors, Forskolin, Arachidonic acid, VPA, BDNF, BT13, O-LPA, GW7646, and Sodium pyruvate. In this model, we focused on maximum expression of PVALB gene expressed in PVALB (+) interneurons (Nahar et al. (2021) Front. Psych. 12:679960). When the model was optimized for maximum expression of PVALB, Forskolin, GW7646, Sodium Pyruvate had the highest factor contributions, 3.14, 27.2, and 17.1 respectively (FIG. 25). The Sodium pyruvate had a significant effect on PVALB expression, so it is included in the stage 5 recipe.

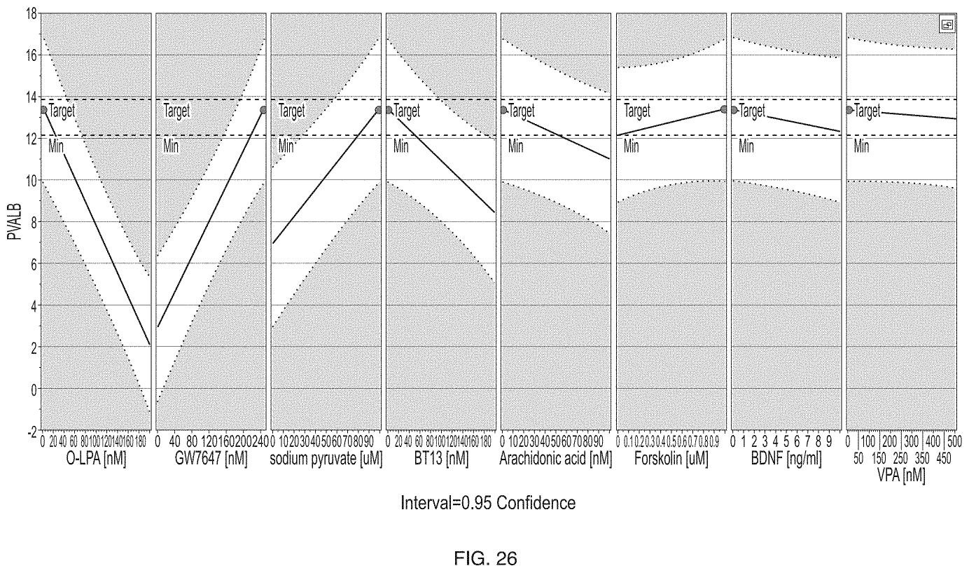

Dynamic profile analysis was used to find common factors with positive impact on expression of PVALB (FIG. 26). It was observed GW7646, Sodium pyruvate, and Forskolin had similar effect on expression of PVALB gene, while BT13 had a significant negative impact on PVALB gene expression. So, it was excluded in the recipe.

### Example 5: Factor Criticality Analysis of Forebrain Neural Progenitor Derived Interneuron-Inducing Culture Conditions

To assess the impact of the elimination of each validated factor, dynamic profile analysis was used and compared the expression level of genes of interest in absence of each finalized factor while others are present. Since expression levels of genes of interest reveal whether the desired outcome is reachable, this factor criticality analysis revealed the extent of importance of each input effector.

To assess the impact of elimination of each validated factor, we again used dynamic profile analysis and compared the expression level of genes of interest in the absence of each finalized factor while others are present. Since expression levels of genes of interest reveal whether the desired outcome is reachable, this factor criticality analysis revealed the extent of importance of each input effector.

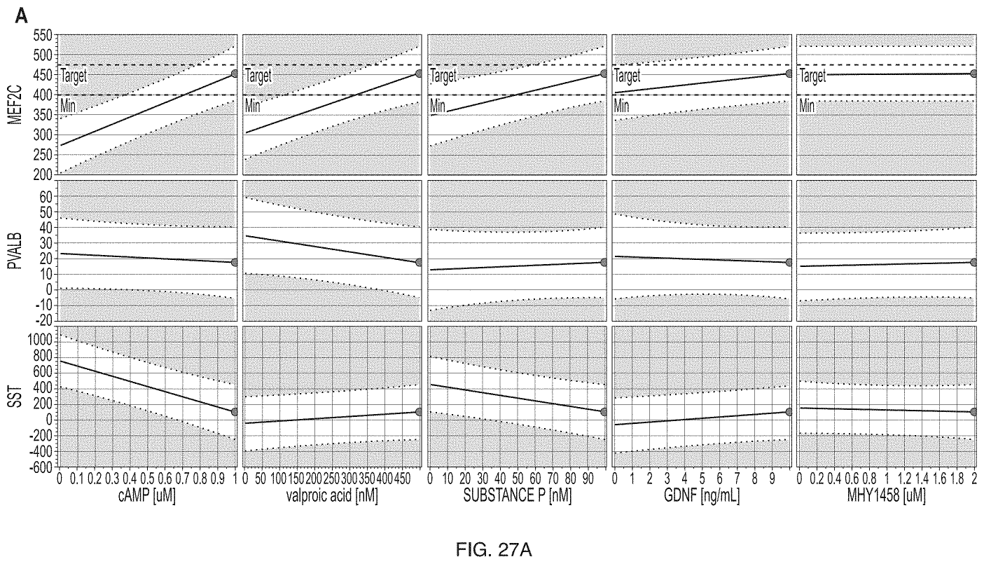

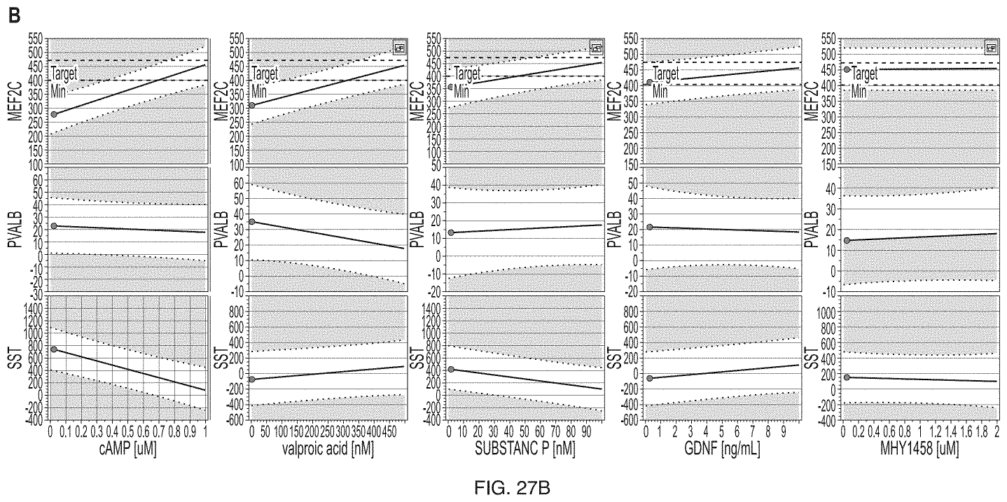

In the stage 4 recipe, each of five finalized factors were removed while the other four factors were present and expression levels of forebrain genes were assessed compared to the presence of all 5 factors. The results are shown in FIG. 27. MEF2C was reported to determine the PVALB-precursors cell fate (Mayer et al. (2018) Nature 555:457-462). We optimize MEF2C expression at this stage. C AMP and Substance P were found to increase the MEF2C expression and decrease the somatostatin expression, so they are included in the stage 4 recipe. GDNF and Valproic acid were found to contribute to PVALB expression but minimal increase somatostatin expression, so they are also included in the stage 4 recipe. MHY1458 was found to increase PVALB expression, and it is included in the recipe. (FIG. 27).

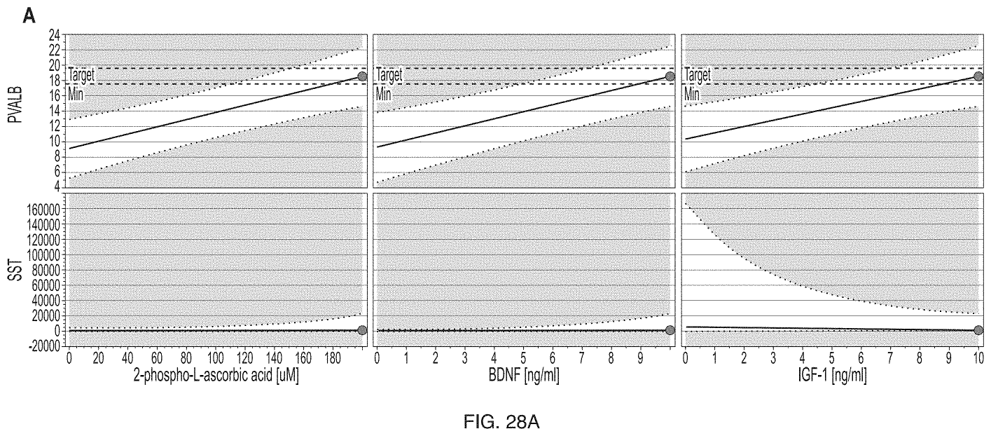

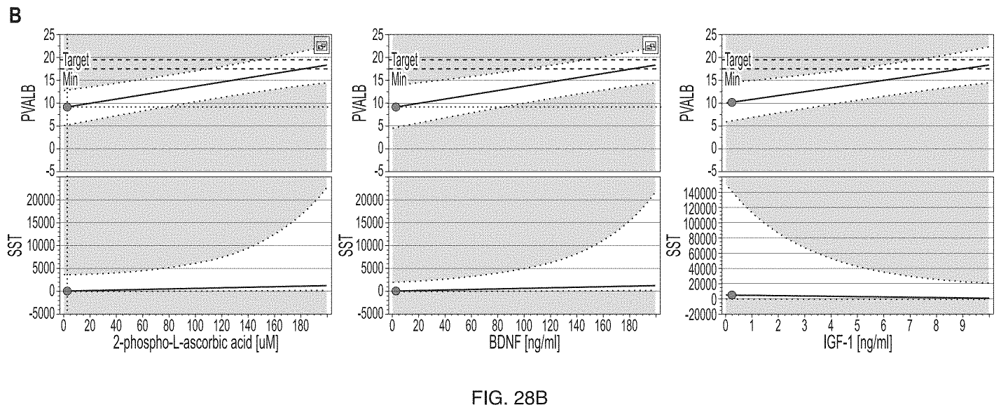

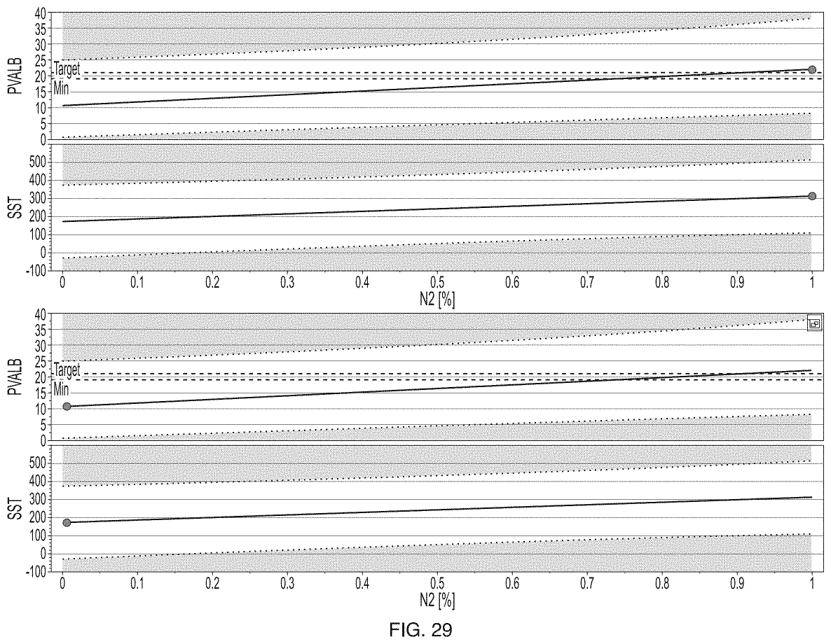

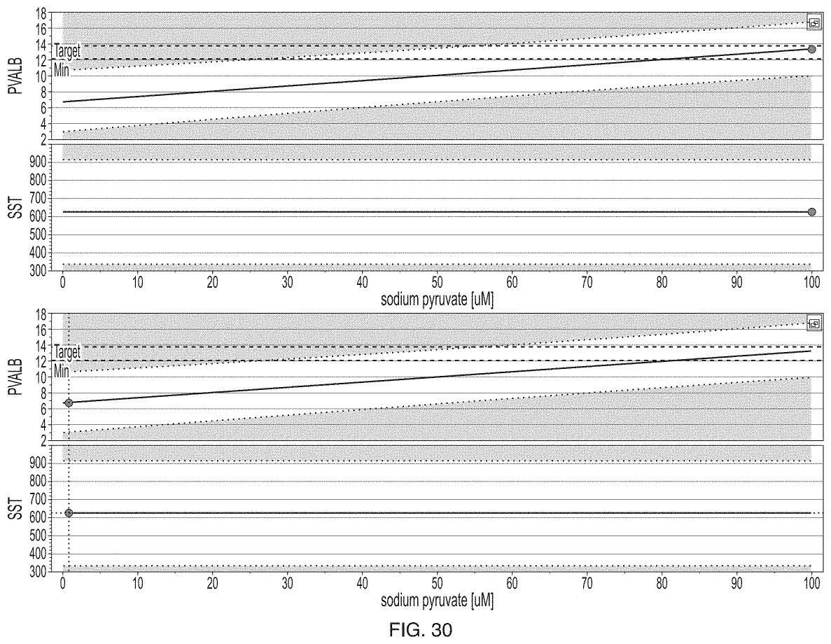

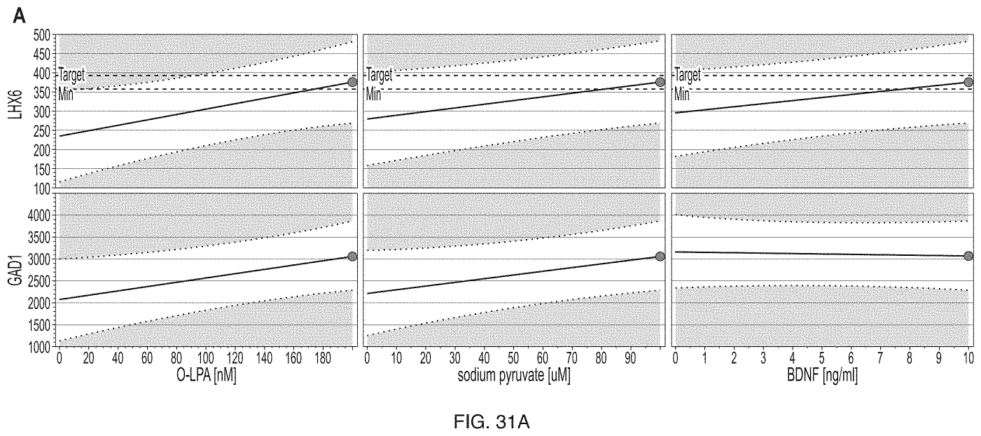

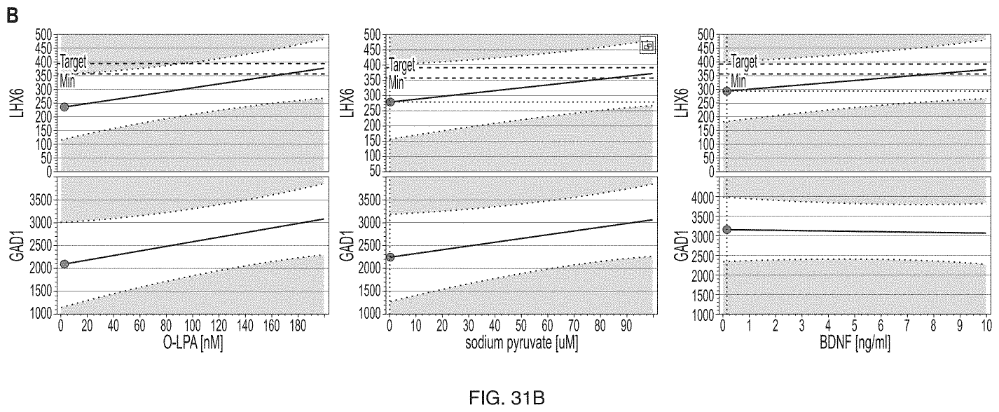

In the stage 5 recipe, each of seven finalized factors were removed while other factors were kept present and expression levels of PVALB and SST were assessed compared to the presence of all seven factors. In this HD-DoE experiment, 2-phospho-L-Ascorbic acid, BDNF, and IGF-1 made positive contribution to the expression of PVALB, while they did not change the expression of somatostatin that was another interneuron marker. 2-phospho-L-ascorbic acid, BDNF, IGF-1 were included in the stage 5 recipe (FIG. 28). In another HD-DoE experiment, N2 and other effectors were found to contribute to the expression of Parvalbumin. Others were also found to increase the expression of somatostatin that was SST interneuron marker. So, they are excluded from the recipe and N2 was included in the stage 5 recipe (FIG. 29). In the last HD-DoE experiment, Sodium Pyruvate was found to contribute to PVALB expression. Sodium pyruvate was included in the stage 5 recipe (FIG. 30). Finally, O-LPA was found to contribute to the expression of LHX6 and GAD1, so it was included in the recipe (FIG. 31).

### Example 6: Immunocytochemistry Validation of Forebrain Neural Progenitor Derived Interneurons Expressing Parvalbumin

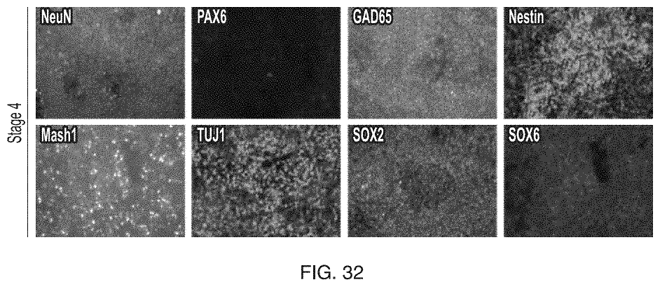

To validate the developed recipes in Example 4, cells first were treated with stage 1, stage 2 and stage 3 differentiation media according to Example 1 and then with stage 4 and stage differentiation media according to Example 4, and immunocytochemistry was used to assess biomarkers of forebrain neurons and neural progenitors at the end of each stage. Biomarkers included MAP2, NeuN, Neurofilament, beta III-tubulin a pan neuronal marker, PAX6, a LGE marker (Kioussi et al. (1999) Proc. Natl. Acad. Sci. 96:14378-14382; Kioussi et al. (1999) Proc. Natl. Acad. Sci. 96:14378-14382), SOX2 a neuronal progenitor marker expressed in ventral forebrain (Hansen et al. (2013) Nature Neurosci. 16:1576-1587), LHX6, a cortical interneuron marker, MASH1 (ASCL1), GFAP a glial marker, SOX6 a marker expressed in post-mitotic progenitors of MGE region (Batista-Brito et al. (2009) Neuron 63:466-481) and GABA a marker specifically expressed by GABAergic interneurons (FIG. 32).

Immunocytochemistry images confirmed the expression of NeuN (Gusel'Nikova et al. (2015) Acta Naturae 7:42-47) by the end of stage 4 in more than 90% of the cells and PAX6 was not detected. The expression of Mash1 and SOX6, which was observed in most of the cultured cells, confirmed the ventral regionalization of the differentiating cells (FIG. 32). Detection of GAD65 in most of the cells confirmed the cells are interneurons.

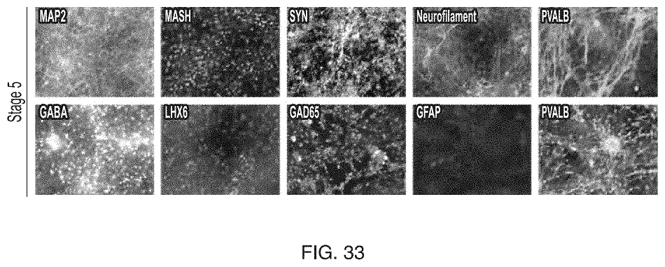

By day 27, which is the end of stage 5, GABA, MASH1, and LHX6 were detected in most of the cells in culture and MAP2, Neurofilament, and Parvalbumin were detected in more than half the culture that confirmed the MGE region of forebrain of cells to differentiated PVALB (+) interneurons (FIG. 33). We also detected Neurofilament, Synapsin and GAD65 in most of the cells in culture that showed these cells have the function of matured neurons (Nguyen et al. (2014) J. Neurosci. 34:14948-14960) (FIG. 33).

Detection of matured neural differentiation markers and absence of dorsal forebrain markers in differentiated cells at 27 days, confirmed the efficiency and robustness of generated recipes for 2-stage differentiation protocol for MGE-committed neural progenitors to differentiated Parvalbumin (+) interneurons.

### EQUIVALENTS

Those skilled in the art will recognize, or be able to ascertain using no more than routine experimentation, many equivalents of the specific embodiments of the invention described herein. Such equivalents are intended to be encompassed by the following claims

1. A method of generating human OTX2+FEZF2+SIX3+ forebrain neural stem cells (FB-NSCs) comprising: culturing human pluripotent stem cells in a culture media comprising a BMP pathway antagonist, a MEK pathway antagonist, a WNT pathway antagonist, an AKT pathway antagonist, an SHH pathway agonist and a PKC pathway antagonist on days 0-3 to obtain human OTX2+FEZF2+SIX3+FB-NSCs.

2. The method of claim 1, wherein the FB-NSCs are further cultured on days 3-6 in a culture media comprising a BMP pathway antagonist, a MEK pathway antagonist, a WNT pathway antagonist and an SHH pathway agonist and lacking an AKT pathway antagonist and a PKC pathway antagonist to obtain human NKX2-1+ ventral forebrain neural stem cells (VFB-NSCs).

3. The method of claim 2, wherein the VFB-NSCs are further cultured on days 6-9 in a culture media comprising a TAK1 pathway antagonist, an SHH pathway agonist, a TGF-β pathway antagonist, a TRK pathway antagonist, a Notch pathway antagonist and an IGF1 pathway agonist to obtain human ASCL1+ medial ganglionic eminence neural progenitor cells (MGE-NPCs).

4. The method of claim 3, wherein the MGE-NPCs are further cultured on days 9-12 in a culture media comprising a CREB or PKA pathway agonist, valproic acid or analog, Substance P or analog, a GDNF pathway agonist and an mTOR pathway agonist to obtain human immatured neurons.

5. The method of claim 4, wherein the human immatured neurons are further cultured on days 12-26 in a culture media comprising a BDNF pathway agonist, an IGF-1 pathway agonist, ascorbic acid or analog, sodium pyruvate or analog, LPA or analog, an N2 supplement and an NEAA supplement to obtain mature parvalbumin+interneurons.

6. The method of claim 1, wherein the human pluripotent stem cells are induced pluripotent stem cells (iPSCs).

7. The method of claim 1, wherein the human pluripotent stem cells are embryonic stem cells.

8. The method of claim 1, wherein the human pluripotent stem cells are attached to vitronectin-coated plates during culturing.

9. The method of claim 1, wherein the BMP pathway antagonist is selected from the group consisting of LDN193189, DMH1, DMH2, Dorsopmorphin, K02288, LDN214117, LDN212854, folistatin, ML347, Noggin, and combinations thereof.

10. The method of claim 1, wherein the MEK pathway antagonist is selected from the group consisting of PD0325901, Binimetinib (MEK162), Cobimetinib (XL518), Selumetinib, Trametinib (GSK1120212), CI-1040 (PD-184352), Refametinib, ARRY-142886 (AZD-6244), PD98059, U0126, BI-847325, RO 5126766, and combinations thereof.

11. The method of claim 1, wherein the WNT pathway antagonist is selected from the group consisting of XAV939, ICG001, Capmatinib, endo-IWR-1, IWP-2, IWP-4, MSAB, CCT251545, KY02111, NCB-0846, FH535, LF3, WIKI4, Triptonide, KYA1797K, JW55, JW 67, JW74, Cardionogen 1, NLS-StAx-h, TAK715, PNU 74654, iCRT3, WIF-1, DKK1, and combinations thereof.

12. The method of claim 1, wherein the SHH pathway agonist is selected from the group consisting of Purmorphamine, GSA 10, SAG, and combinations thereof.

13. The method of claim 1, wherein the AKT pathway antagonist is selected from the group consisting of MK2206, GSK690693, Perifosine (KRX-0401), Ipatasertib (GDC-0068), Capivasertib (AZD5363), PF-04691502, AT 7867, Triciribine (NSC154020), ARQ751, Miransertib (ab235550), Borussertib, Cerisertib, and combinations thereof.

14. The method of claim 1, wherein the PKC pathway antagonist is selected from the group consisting of Go 6983, Sotrastaurin, Enzastaurin, Staurosporine, LY31615, Go 6976, GF 109203X, Ro 31-8220 Mesylate, and combinations thereof.

15. The method of claim 3, wherein the TAK1 pathway antagonist is selected from the group consisting of Takinib, Dehydoabietic acid, NG25, Sarsasapogenin, and combinations thereof.

16. The method of claim 3, wherein the TGFβ pathway antagonist is selected from the group consisting of A 83-01, SB-431542, GW788388, SB525334, TP0427736, RepSox, SD-208, and combinations thereof.

17. The method of claim 3, wherein the TRK pathway antagonist is selected from the group consisting of GNF-5837, BMS-754807, UNC2020, Taletrectinib, Altiratinib, Selitrectinib, PF 06273340, and combinations thereof.

18. The method of claim 3, wherein the Notch pathway antagonist is selected from the group consisting of GSI-XX, RO4929097, Semagacestat, Dibenzazepine, LY411575, Crenigacestat, IMR-1, IMR-1A, FLI-06, DAPT, Valproic acid, YO-01027, CB-103, Tangeretin, BMS-906024, Avagacestat, Bruceine D, and combinations thereof.

19. The method of claim 3, wherein the IGF1 pathway agonist is selected from the group consisting of IGF1, IGF1-Ado, X10, mecasermin, and combinations thereof.

20. The method of claim 4, wherein the CREB or PKA pathway agonist is selected from the group consisting of cAMP, Dibutyryl-cAMP, 8-Br-cAMP, cAMPS-Sp, CW 008, Forskolin, 8-CPT-cAMP, CW 008, Adenosine 3′,5′-cyclic Monophosphate, N6-Benzoyl-, Sodium Salt, Adenosine 3′,5′-cyclic monophosphate sodium salt monohydrate, (S)-Adenosine, cyclic 3prime,5prime-(hydrogenphosphorothioate) triethylammonium, Sp-Adenosine 3prime,5prime-cyclic monophosphorothioate triethylammonium salt, Sp-5,6-DCI-cBiMPS, 8-Bromoadenosine 3′,5′-cyclic Monophosphothioate, Sp-Isomer sodium salt, Adenosine 3prime,5prime-cyclic Monophosphorothioate,8-Bromo-, Sp-Isomer, Sodium Salt, Sp-8-pCPT-cyclic GMPS Sodium, 8-Bromoadenosine 3′,5′-cyclic monophosphate, N6-Monobutyryladenosine 3prime:5prime-cyclic monophosphate sodium salt, 8-PIP-cAMP, Sp-cAMPS, and combinations thereof.

21. The method of claim 4, wherein valproic acid or analog is selected from the group consisting of valproic acid, valproate, sodium valproate and valproate semisodium.

22. The method of claim 4, wherein Substance P is present in the culture media at a concentration within a range of 100-150 nM.

23. The method of claim 4, wherein the GDNF pathway agonist is selected from the group consisting of GDNF, BT13, BT44, and combinations thereof.

24. The method of claim 4, wherein the mTOR pathway agonist is selected from the group consisting of MHY1458, NV-5138, Testosterone, 3-benzyl-5-((2-nitrophenoxy) methyl)-dihydrofuran-2(3H)-one (3BDO), 3BDO, L-leucine, NV-5138 hydrochloride, NV-5138, L-leucine-d1, L-leucine-2-13C,15N, Leucine-13C6, L-leucine-d7, L-leucine-d10, L-leucin-d2, 1-leucine-d3, L-leucine-18O2, L-leucine-13C, L-leucine-2-13C, L-leucine-13C6-15N, L-leucine-15N, L-leucine-1-13C,15N, and combinations thereof.

25. The method of claim 5, wherein the BDNF pathway agonist is selected from the group consisting of BDNF, rotigotine, 7, 8-DHF, ketamine, tricyclic dimeric peptide-6 (TDP6), LM22A-4, and combinations thereof.

26. The method of claim 5, wherein ascorbic acid or analog thereof is selected from the group consisting of vitamin C, 2-phospho-L-ascorbic acid, L-ascorbic acid, sodium ascorbyl phosphate, magnesium ascorbyl phosphate, ascorbyl glucoside, tetrahexyldecyl ascorbate (THD), ethylated L-ascorbic acid, and combinations thereof.

27. The method of claim 5, wherein sodium pyruvate is present in the culture media at a concentration of 100-300 μM.

28. The method of claim 5, wherein LPA or analog thereof is selected from the group consisting of lysophosphatidic acid, 2-[[3-(1,3-dioxo-1H-benz[de]isoquinolin-2(3H)-yl)propyl]thio]benzoic acid, 1-Oleoyl lysophosphatidic acid sodium salt, UCM-05194, and combinations thereof.

29. The method of claim 5, wherein the N2 supplement is present in the culture media at a concentration of 1%.

30. The method of claim 5, wherein the NEAA supplement is present in the culture media at a concentration of 1%.

31. A method of generating human NKX2-1 ventral forebrain neural stem cells (VFB-NSCs) comprising:
(a) culturing human pluripotent stem cells in a culture media comprising a BMP pathway antagonist, a MEK pathway antagonist, a WNT pathway antagonist, an AKT pathway antagonist, an SHH pathway agonist and a PKC pathway antagonist on days 0-3 to obtain human OTX2+FEZF2+SIX3+ forebrain neural stem cells (FB-NSCs); and
(b) further culturing the human OTX2+FEZF2+SIX3+FB-NSCs on days 3-6 in a culture media comprising a BMP pathway antagonist, a MEK pathway antagonist, a WNT pathway antagonist and an SHH pathway agonist and lacking an AKT pathway antagonist and a PKC pathway antagonist to obtain human NKX2-1+ ventral forebrain neural stem cells (VFB-NSCs).

32. The method of claim 31, wherein LDN193189 is present in the culture media at a concentration of 275 nM in step (a) and 250 nM in step (b), PD0325901 is present in the culture media at a concentration of 110 nM in step (a) and 100 nM in step (b), XAV939 is present in the culture media at a concentration of 110 nM in step (a) and 100 nM in step (b), Purmorphamine is present in the culture media at a concentration of 550 nM in step (a) and 500 nM in step (b), MK2206 is present in the culture media in step (a) at a concentration of 138 nM, and Go 6983 is present in the culture media in step (a) at a concentration of 110 nM.

33. A method of generating human ASCL1+ medial ganglionic eminence neural progenitor cells (MGE-NPCs) comprising:
(a) culturing human pluripotent stem cells in a culture media comprising a BMP pathway antagonist, a MEK pathway antagonist, a WNT pathway antagonist, an AKT pathway antagonist, an SHH pathway agonist and a PKC pathway antagonist on days 0-3 to obtain human OTX2+FEZF2+SIX3+ forebrain neural stem cells (FB-NSCs);
(b) further culturing the human OTX2+FEZF2+SIX3+FB-NSCs on days 3-6 in a culture media comprising a BMP pathway antagonist, a MEK pathway antagonist, a WNT pathway antagonist and an SHH pathway agonist and lacking an AKT pathway antagonist and a PKC pathway antagonist to obtain human NKX2-1+ ventral forebrain neural stem cells (VFB-NSCs); and
(c) further culturing the human NKX2-1+VFB-NSCs on days 6-9 in a culture media comprising a TAK1 pathway antagonist, an SHH pathway agonist, a TGF-β pathway antagonist, a TRK pathway antagonist, a Notch pathway antagonist and an IGF1 pathway agonist to obtain human ASCL1+ medial ganglionic eminence neural progenitor cells (MGE-NPCs).

34. The method of claim 33, wherein LDN193189 is present in the culture media at a concentration of 275 nM in step (a) and 250 nM in step (b), PD0325901 is present in the culture media at a concentration of 110 nM in step (a) and 100 nM in step (b), XAV939 is present in the culture media at a concentration of 110 nM in step (a) and 100 nM in step (b), Purmorphamine is present in the culture media at a concentration of 550 nM in step (a) and 500 nM in step (b), MK2206 is present in the culture media in step (a) at a concentration of 138 nM, Go 6983 is present in the culture media in step (a) at a concentration of 110 nM, Takinib is present in the culture media in step (c) at a concentration of 2 uM, A 83-01 is present in the culture media in step (c) at a concentration of 500 nM, GNF-5837 is present in the culture media in step (c) at a concentration of 50 nM, GSI-XX is present in the culture media in step (c) at a concentration of 100 nM and IGF-1 is present in the culture media in step (c) at a concentration within a range of ng/ml.

35. A method of generating human mature parvalbumin+interneurons from human medial ganglionic eminence neural progenitor cells (MGE-NPCs) comprising:
(a) culturing human MGE-NPCs in a culture media comprising a CREB or PKA pathway agonist, valproic acid or analog, Substance P or analog, a GDNF pathway agonist and an mTOR pathway agonist on days 0-3 to obtain human immatured neurons; and
(b) culturing the human immatured neurons in a culture media comprising a BDNF pathway agonist, an IGF-1 pathway agonist, ascorbic acid or analog, sodium pyruvate or analog, LPA or analog, an N2 supplement and an NEAA supplement on days 3-17 to obtain human mature parvalbumin+interneurons.

36. The method of claim 35, wherein cAMP is present at a concentration of 1.0 μM, valproic acid is present at a concentration of 500 nM, Substance P is present at a concentration of 100 nM, GDNF is present at a concentration of 10 ng/ml, MHY1458 is present at a concentration of 2 μM, BDNF is present at a concentration of 10 ng/ml, IGF-1 is present at a concentration of 10 ng/ml, 2-phospho-L-ascorbic acid is present at a concentration of 200 μM, sodium pyruvate is present at a concentration of 100 μM, O-LPA is present at a concentration of 200 μM, N2 supplement is present at a concentration of 1.0% and NEAA supplement is present at a concentration of 1.0%.

37. A culture media for obtaining human OTX2+FEZF2+SIX3+ forebrain neural stem cells (FB-NSCs) according to the method of claim 1, comprising a BMP pathway antagonist, a MEK pathway antagonist, a WNT pathway antagonist, an AKT pathway antagonist, an SHH pathway agonist and a PKC pathway antagonist.

38. A culture media for obtaining human NKX2-1+ ventral forebrain neural stem cells (VFB-NSCs) according to the method of claim 2, comprising a BMP pathway antagonist, a MEK pathway antagonist, a WNT pathway antagonist and an SHH pathway agonist.

39. A culture media for obtaining human ASCL1+ medial ganglionic eminence neural progenitor cells (MGE-NPCs) according to the method of claim 3, comprising a TAK1 pathway antagonist, an SHH pathway agonist, a TGF-β pathway antagonist, a TRK pathway antagonist, a Notch pathway antagonist and an IGF1 pathway agonist.

40. A culture media for obtaining human immatured neurons according to the method of claim 4, comprising a CREB or PKA pathway agonist, valproic acid or analog, Substance P or analog, a GDNF pathway agonist and an mTOR pathway agonist.

41. A culture media for obtaining human mature GABAergic interneurons according to the method of claim 5, comprising a CREB or PKA pathway agonist, valproic acid or analog, Substance P or analog, a GDNF pathway agonist and an mTOR pathway agonist.

42. An isolated cell culture of human OTX2+FEZF2+SIX3+ forebrain neural stem cells (FB-NSCs) prepared according to the method of claim 1, the culture comprising human OTX2+FEZF2+SIX3+FB-NPCs cultured in a culture media comprising a BMP pathway antagonist, a MEK pathway antagonist, a WNT pathway antagonist, an AKT pathway antagonist, an SHH pathway agonist and a PKC pathway antagonist.

43. An isolated cell culture of human NKX2-1+ ventral forebrain neural stem cells (VFB-NSCs) prepared according to the method of claim 2, the culture comprising human NKX2-1+VFB-NPCs cultured in a culture media comprising a BMP pathway antagonist, a MEK pathway antagonist, a WNT pathway antagonist and an SHH pathway agonist.

44. An isolated cell culture of human ASCL1+ medial ganglionic eminence neural progenitor cells (MGE-NPCs) prepared according to the method of claim 3, the culture comprising human ASCL1+MGE-NPCs cultured in a culture media comprising a TAK1 pathway antagonist, an SHH pathway agonist, a TGF-β pathway antagonist, a TRK pathway antagonist, a Notch pathway antagonist and an IGF1 pathway agonist.

45. An isolated cell culture of human immatured neurons prepared according to the method of claim 4, the culture comprising human immatured neurons cultured in a culture media comprising a CREB or PKA pathway agonist, valproic acid or analog, Substance P or analog, a GDNF pathway agonist and an mTOR pathway agonist.

46. An isolated cell culture of human mature GAB Aergic interneurons prepared according to the method of claim 5, the culture comprising human mature GABAergic interneurons cultured in a culture media comprising a BDNF pathway agonist, an IGF-1 pathway agonist, ascorbic acid or analog, sodium pyruvate or analog, LPA or analog, an N2 supplement and an NEAA supplement.

47. Human OTX2+FEZF2+SIX3+ forebrain neural stem cells (FB-NSCs) generated by the method of claim 1.

48. Human NKX2-1+ ventral forebrain neural stem cells (VFB-NSCs) generated by the method of claim 2.

49. Human ASCL1+ medial ganglionic eminence neural progenitor cells (MGE-NPCs) generated by the method of claim 3.

50. Human immatured neurons generated by the method of claim 4.

51. Human parvalbumin+mature GABAergic interneurons generated by the method of claim 5.
